(19) United States
(12) Patent Application Publication
(10) Pub. No.: US 2014/0236265 A1
(43) Pub. Date: Aug. 21, 2014

(54) COMPOSITIONS, METHODS, DEVICES, AND SYSTEMS FOR SKIN CARE

(71) Applicant: TRIA Beauty, Inc., Dublin, CA (US)

(72) Inventors: MIchael P. O'Neil, Dublin, CA (US); Joel Rubin, East Haven, CT (US); Drake Stimson, Terrace Park, OH (US)

(21) Appl. No.: 14/183,452

(22) Filed: Feb. 18, 2014

Related U.S. Application Data
(63) Continuation of application No. 12/554,872, filed on Sep. 4, 2009, now abandoned.
(60) Provisional application No. 61/097,513, filed on Sep. 16, 2008.

(51) Int. Cl. A61K 8/365; A61K 8/92; A61Q 19/00; A61K 8/66; A61K 8/67; A61K 8/31; A61N 5/06; A61Q 19/10

(52) U.S. Cl. CPC A61K 8/365; A61N 5/0616; A61K 8/922; A61Q 19/10; A61K 8/66; A61K 8/673; A61K 8/31; A61Q 19/00; A61N 2005/0663
USPC 607/88

(57) ABSTRACT

Accordingly, a need has arisen for improved compositions, methods, devices, and systems for skin care including, without limitation, topical compositions and phototherapy devices and methods. A skin care method may comprise, in some embodiments, contacting at least a portion of the skin of a subject with a homogeneous, stable, self-foaming composition, illuminating the at least a portion of the skin exclusively with light of a desired wavelength (e.g., from about 390 nm to about 430 nm, and contacting the at least a portion of the skin with an anti-oxidant serum composition.

### CROSS-REFERENCE TO RELATED APPLICATIONS

This application claims priority to U.S. Provisional Application No. 61/097,513 filed Sep. 14, 2008 (Applicant Ref. No. TBI-1010P), the entire contents of which are hereby incorporated in their entirety by this reference. This application claims priority to U.S. application Ser. No. 12/554,831 filed Sep. 4, 2009, the entire contents of which are hereby incorporated in their entirety by this reference.

### FIELD OF THE DISCLOSURE

The present disclosure relates, in some embodiments, to compositions, methods, devices, and systems for skin care.

### BACKGROUND OF THE DISCLOSURE

Acne may be caused by one or more of (i) excess sebum production, (ii) hyperkeritinization, (iii) excessive bacterial (e.g., P. acnes) load within the pilosebaceous unit, and (iv) a heightened overall inflammatory response. Oral Accutane™ (isotretinoin) therapy may be used to eliminate (e.g., completely) sebum production, but toxicity and side effects including birth defects may detract from its use.

### SUMMARY

Accordingly, a need has arisen for improved compositions, methods, devices, and systems for skin care including, without limitation, topical compositions and phototherapy devices and methods.

The present disclosure relates, according to some embodiments, to compositions, methods, devices, and systems for skin care including, without limitation, topical compositions and phototherapy devices and methods for acne care.

A skin care method may comprise, in some embodiments, contacting at least a portion of the skin of a subject with a homogeneous, stable, self-foaming composition, illuminating the at least a portion of the skin exclusively with light of a desired wavelength (e.g., from about 390 nm to about 430 nm, and contacting the at least a portion of the skin with an anti-oxidant serum composition. A skin care method may comprise, in some embodiments, rinsing off (e.g., completely rinsing off) the self-foaming composition prior to the illuminating. According to some embodiments, a self-foaming composition may comprise (a) an effective exfoliating amount of at least one of an alpha hydroxy acid and a beta hydroxy acid, (b) a saturated dicarboxylic acid, and (c) a sulfate ester of coconut oil. For example, a self-foaming composition may comprise up to about 10% (w/w) an alpha hydroxy acid. A nonlimiting example of an alpha hydroxy acid is glycolic acid. For example, a self-foaming composition may comprise from about 0.5% (w/w) to about 5.5% (w/w) of a beta hydroxy acid. A nonlimiting example of an beta hydroxy acid is salicylic acid. A saturated dicarboxylic acid may comprise, in some embodiments azelaic acid (e.g., from about 0.8% (w/w) to about 1.2% (w/w)). According to some embodiments, a sulfate ester of coconut oil may comprises sodium coco-sulfate (e.g., from about 3% (w/w) to about 5% (w/w)). A self-foaming composition further comprises cocamidopropyl betaine (e.g., up to about 7.5% (w/w) or over about 7.5% (w/w)) in some embodiments. A self-foaming composition may comprise, according to some embodiments, menthyl lactate (e.g., from about 0.4% (w/w) to about 0.6% (w/w)).

In some embodiments, illuminating may comprise illuminating the skin exclusively with light having a wavelength from about 407 nm to about 420 nm in some embodiments. Illuminating may comprise, in some embodiments, illuminating at a desired or required intensity. For example, the illuminating may comprise illuminating the at least a portion of the skin at a light intensity of about 400 mW/cm².

An anti-oxidant serum may comprise, according to some embodiments, superoxide dismutase. In some embodiments, an anti-oxidant composition may comprise a beta hydroxy acid (e.g., from about 0.3% (w/w) to about 2.2% (w/w)). A nonlimiting example of an beta hydroxy acid is salicylic acid.

The present disclosure relates, in some embodiments, to homogeneous, stable, self-foaming composition. For example, a homogeneous, stable, self-foaming composition may comprise (a) an effective exfoliating amount of at least one of an alpha hydroxy acid (e.g., up to about 10% (w/w)) and a beta hydroxy acid (e.g., up to about 5% (w/w)), (b) a saturated dicarboxylic acid (e.g., from about 0.8% (w/w) to about 1.2% (w/w)); and (c) a sulfate ester of coconut oil (e.g.,). A nonlimiting example of an alpha hydroxyl acid is glycolic acid. A nonlimiting example of a beta hydroxyl acid is salicylic acid. A nonlimiting example of a saturated dicarboxylic acid is azelaic acid. A nonlimiting example of a sulfate ester of coconut oil may comprise sodium coco-sulfate. According to some embodiments, a homogeneous, stable, self-foaming composition may comprise cocamidopropyl betaine (e.g., up to about 7.5% (w/w/) or over about 7.5% (w/w)). A homogeneous, stable, self-foaming composition may comprise, in some embodiments, menthyl lactate (e.g., from about 0.4% (w/w) to about 0.6% (w/w)).

The present disclosure relates, according to some embodiments, to a phototherapy kit, for example, comprising a self-foaming cleanser, a phototherapy device configured to illuminate a tissue with light of a desired wavelength, and an anti-oxidant serum. In some embodiments, a phototherapy kit may comprise a homogeneous, stable, cleanser composition comprising an effective exfoliating amount of at least one of an alpha hydroxy acid and a beta hydroxy acid (optionally azelaic acid); a phototherapy device configured to emit light having a wavelength of from about 390 nanometers to about 430 nanometers toward a target portion of a subject's skin; an anti-oxidant composition comprising superoxide dismutase; and instructions for applying the cleanser composition to the target portion of a subject's skin, illuminating the target portion of the subject's skin with the phototherapy device, and applying the anti-oxidant composition to the target portion of the subject's skin.

The present disclosure further relates, in some embodiments, to a phototherapy device. A phototherapy device may comprise, for example, a housing; a light source in the housing and configured to emit light having a wavelength of from about 390 nanometers to about 430 nanometers toward a target portion of the subject; an optical filter interposed between the light source and the target portion of the subject and configured to reduce or eliminate light having a wavelength less than about 390 nanometers and/or light having a wavelength over about 430 nanometers; a power supply; a touch-sensitive switch configured to electrically couple the power supply and the light source upon contact with a subject's skin and to electrically uncouple the power supply and the light source when not in contact with a subject's skin; a light shield configured to contact the subject's skin and reduce or eliminate exposure of non-target portions of the subject to emitted light; an optical mixer and a diffuser interposed between the light source and the target portion of the subject; and/or an output window interposed between the optical mixer and the subject. A phototherapy device may comprise, in some embodiments, an optical filter configured to filter light having a wavelength less than about 407 nanometers and filter light having a wavelength over about 420 nanometers. According to some embodiments, a phototherapy device may comprise a light source comprises a light emitting diode, a laser diode, a flashlamp, or combinations thereof. An optical mixer may comprise polymethyl methacrylate (acrylic), glass, quartz, or combinations thereof, in some embodiments. An output window may comprise, according to some embodiments, glass, sapphire, quartz, diamond, or combinations thereof

### BRIEF DESCRIPTION OF THE DRAWINGS

Some embodiments of the disclosure may be understood by referring, in part, to the present disclosure and the accompanying drawings, wherein:

FIG. 1 illustrates a system for visually identifying the affected areas on a patient, and for subsequently treating those areas according to a specific example embodiment of the disclosure;

FIG. 2 illustrates in greater detail a device for visualizing the affected areas on a patient according to a specific example embodiment of the disclosure;

FIG. 3 illustrates an embodiment of a device for treating affected areas on a patient according to a specific example embodiment of the disclosure;

FIG. 4 illustrates in an exploded perspective view the treatment device of FIG. 3;

FIG. 5 illustrates in greater detail the light source of the treatment device of FIG. 3 and FIG. 4;

FIG. 6 illustrates in schematic diagram form an embodiment of the circuitry of the treatment device shown in FIG. 3 and FIG. 4;

FIG. 7 illustrates in greater detail the airflow venting of the treatment device of FIG. 3 and FIG. 4;

FIG. 8 illustrates in flow diagram form a process for treating acne according to a specific example embodiment of the disclosure;

FIG. 9 illustrates a second embodiment of a device for treating affected areas on a patient.

FIG. 10 illustrates the end of the treatment device of FIG. 9 opposite the outlet window and shows the aperture for inserting a removable timing cartridge.

FIGS. 11A-11D illustrate an exploded perspective view of the treatment device of FIG. 9 and FIG. 10.

FIG. 12 illustrates in greater detail the light source of the treatment device of FIG. 9 and FIG. 10.

FIG. 13 illustrates in greater detail the air intake and outlet venting of the treatment device of FIG. 9 and FIG. 10.

FIG. 14 illustrates in schematic diagram form an embodiment of the circuitry of the treatment device shown in FIG. 9 and FIG. 10.

FIGS. 15, 16A, and 16B illustrate, respectively, the display window of the device of FIG. 9 and FIG. 10, the display window when the timing cartridge is full, and the display window when the timing cartridge is fully discharged.

FIG. 17 illustrates in flow diagram form an embodiment of a process for treating acne in accordance with the present invention.

FIG. 18 is a schematic illustration of one embodiment of the invention.

FIG. 19 is a graphical illustration of the results of a skin temperature calculation for a first set of conditions.

FIG. 20 is a graphical illustration of the results of a skin temperature calculation for a second set of conditions.

FIG. 21 is a graphical illustration of the results of a skin temperature calculation for a third set of conditions.

FIG. 22 is a graphical illustration of the results of a skin temperature calculation for a fourth set of conditions.

FIG. 23 is a schematic illustration of one embodiment of a light source comprising light emitting diodes which is suitable for use in the invention.

FIG. 24 illustrates blemish reduction over time achieved with a blue light device according to a specific example embodiment of the disclosure or with benzyol peroxide (BPO); and

FIG. 25 illustrates a cleanser-phototherapy-anti-oxidant regime according to a specific example embodiment of the disclosure.

### DETAILED DESCRIPTION

The present disclosure relates, according to some embodiments, to compositions, methods, devices, and systems for skin care including, without limitation, topical compositions and phototherapy devices and methods for acne care.

Acne may be caused by one or more of (i) excess sebum production, (ii) hyperkeritinization, (iii) excessive bacterial (e.g., P. acnes) load within the pilosebaceous unit, and (iv) a heightened overall inflammatory response. Without limiting any particular embodiment(s) to a specific mechanism of action, compositions, methods, devices, and systems, according to some embodiments, may reduce one or more of factors (ii), (iii), and (iv). For example, compositions, methods, devices, and/or systems may reduce all three of these factors.

### Cleanser Compositions

According to some embodiments, skin may be contacted with a composition (e.g., foaming cleanser) prior to exposure to light (e.g., phototherapy). For example, a cleanser may be applied to remove sunblock and/or makeup which may otherwise impede the effect of phototherapy. A cleanser may be keratolytic without inducing inflammation or dryness, in some embodiments. A cleanser may have, according to some embodiments, any desired or required pH. The pH of a cleanser may be similar to the pH of healthy skin, in some embodiments. A cleanser composition, in some embodiments, may comprise alpha and beta hydroxyl acids and have a pH below neutral.

In addition to excess sebum, acne-inducing bacteria may benefit from and/or require the presence of dead skin cells. For example, acne-inducing bacteria may metabolize dead skin cells to completely colonize a sebaceous gland. Keratin is sticky and may help dead skin cells to work their way back into a gland. Without excess keratin, these cells may more easily and/or completely slough off and fall away. Alpha and/or beta hydroxy acids may remove excess dead surface skin cells and keratin; they are so-called keratolytics. For example, salicylic acid may dissolve keratin, penetrate pores deeply to exfoliate excess keratin, and/or remove keratin plugs (e.g., clogging the pilosebaceous duct). Glycolic acid may promote deep penetration and/or promote healthy skin tone and/or texture. Unlike benzyol peroxide, salicylic and glycolic acids at the concentrations disclosed herein do not induce irritation or inflammation. They may induce a tingling sensation that may suggest to the user that the cleanser may be doing more than just soap would.

According to some embodiments, a cleanser may comprise liposome encapsulated a saturated dicarboxylic acid (e.g., azelaic acid). Azelaic acid may have antimicrobial properties and/or non-inflammatory properties and/or may reduce dyspigmentations. The concentration of a saturated dicarboxylic acid (e.g., azelaic acid) may be from about 0.8% (w/w) to about 1.2% (w/w) in some embodiments.

A cleanser (e.g., an acidic cleanser) may have, according to some embodiments, one or more foaming properties. In some embodiments, a cleanser may comprise a sulfate ester of coconut oil (e.g., sodium coco-sulfate) having the general formula ROSO₃Na, wherein R is an alkyl group. Unlike surfactants such as sodium lauryl sulfate which have fixed alkyl chain lengths (C₁₂ in this case), sodium coco sulfate has a considerable range of chain lengths, from C₁₂ to at least C₁₈. Without limiting any particular embodiment(s) to a specific mechanism of action, compositions, the range of chain lengths may promote generation of a stable foam by allowing a range of surfactant micelle sizes. Under some circumstances, a stable, densely foaming composition may (e.g., advantageously) allow a user to apply it only to an affected area of the skin without the preparation flowing into undesired areas such as the eyes or mouth. A foam may have greater visibility than, for example, a clear fluid.

In some embodiments, a cleanser may comprise cocamidopropyl betaine, for example, at a very high concentration (e.g., as high or higher than about 7.5% (w/w)). This naturally-occurring compound (found in beets) may be used by plant and animal cells to protect against stress and inflammation. It may have other desirable properties. For example, it may improve foam creaminess and volume, improve skin feel, and/or reduce the stickiness of the formulation. It may also help the foam quickly rinse off, which may help avoid blue light absorption by anything other than the skin and/or may be better received by users who dislike anything that leaves residue. Betaine, an osmolyte, may help skin (e.g., skin cells) to retain moisture, making skin smoother and plumper. Since betaine may increase the water solubility of active compounds, formulations with salicylic acid may be prepared and/or used at room temperature, which may reduce active ingredient degradation.

In some embodiments, a cleanser may comprise one or more fragrances. A cleanser may comprise water lily CD-50045, for example. This compound has a light and elegant fragrance that may be unlike the “fruit and flowers” or “menthol and camphor” scents used in other acne preparations. It may also tolerate low pH and/or rinse away completely. A fragrance may serve, according to some embodiments, as a marker for the presence of odorless components. Therefore, complete rinsing of the fragrance may offer the user assurance and/or comfort that the cleanser has been removed completely, leaving no residue, particularly no residue that may be perceived as irritating and/or inflammatory.

A cleanser may comprise, according to some embodiments, a compound that provides a cooling sensation (e.g., to balance any warming sensation that may accompany phototherapy). Such a compound may be desirable, for example, for any type of pain or inflammation. While menthol may be used for this purpose, in some embodiments, its scent may not be welcome or desirable. Menthyl lactate, which has no odor, may have more (e.g., 30 times more) cooling effect than menthol.

In some embodiments, a cleanser composition may have any suitable form for topical application. For example, a cleanser composition may be formulated as a liquid, a cream, a paste, a gel, a jelly, a peel, an ointment, a rub, and/or a powder. A cleanser composition may be applied, according to some embodiments, by wiping, rubbing, smearing, spraying, coating, or otherwise contacting the material with the skin of a subject.

A composition (e.g., an anti-acne foaming cleanser), in some embodiments, may comprise butylene glycol, cocamidopropyl betaine (e.g., over about 7.5% (w/w)), sodium olefin sulfonate (e.g., C14-C16), polysorbate 20, an alpha hydroxy acid (e.g., up to about 5% (w/w)), sodium coco-sulfate, a beta hydroxy acid (e.g., up to about 2% (w/w)), ethoxydiglycerol, water lily CD-50045, ammonium hydroxide, linoleamidopropyl PG-dimonium chloride phosphate, EDTA; e.g., disodium EDTA), menthyl lactate, menthol, methylchloroisothiazolinone, methylisothiazolinone, and/or combinations thereof. The concentration of each component, may be varied, according to some embodiments, as desired or required within a range of ±20% of the amount indicated above and/or the amount shown in Example 1. For example, the concentration of cocamidopropyl betaine may be from about 6% (w/w) to about 9% (w/w). In some embodiments, the concentration of beta hydroxy acid may be from about 0.5% (w/w) to about 3.5% (w/w).

### Anti-Oxidant Compositions

Phototherapy (e.g., in the presence of endogenous bacterial porphyrins) may generate, in some embodiments, intracellular reactive oxygen species (ROS) including, for example, singlet oxygen. While a ROS may be strongly antibacterial, it may also have undesirable effects on skin if left unchecked including oxidative stress and concomitant inflammation within the skin. Thus, in some embodiments, skin subjected to phototherapy may be contacted with an anti-oxidant composition (e.g., to quench ROS), if desired and/or required. According to some embodiments, an anti-oxidant composition may comprise superoxide dismutase (SOD) (e.g., about 0.2% (w/w)). SOD naturally occurs in human skin and may eliminate free superoxide radicals without being expended in the process.

In some embodiments, an anti-oxidant composition may comprise salicylic acid (e.g., about 1.25% (w/w)), which may have keratolytic and/or mild anti-bacterial properties. An anti-oxidant composition may comprise, according to some embodiments, squalene (e.g., about 0.875% (w/w)), which may penetrate and/or moisturize skin. In some embodiments, an anti-oxidant composition may comprise vitamin B niacinamide (e.g., about 0.5% (w/w)), which may reorder the upper layer of the stratum corneum, may reduce age spots and/or blotchiness, may reduce fine lines, and/or may have exfoliating properties. An anti-oxidant composition may comprise, according to some embodiments, vitamin C tetrahexyldecyl ascorbate (e.g., about 0.1% (w/w)), which may have anti-oxidant properties and/or may increase collagen and/or melanin synthesis. In some embodiments, an anti-oxidant composition may comprise vitamin A retinyl palmitate (e.g., about 0.1% (w/w)), which may increase skin thickness, may improve ordering of collagen and elastin fibers, may moisturize skin, and/or may improve skin tone, fine lines and/or wrinkles. An anti-oxidant composition may comprise, according to some embodiments, azelaic acid (e.g., about 0.08% (w/w)), which may have antibacterial, keratinolytic, comedolytic, free-radical-scavenger, and/or anti-inflammatory properties. The concentration of each component, may be varied, according to some embodiments, as desired or required within a range of ±20% of the amount indicated above and/or the amount shown in Example 2. For example, the concentration of vitamin B niacinamide may be from 0.4% (w/w) to about 0.6% (w/w). In some embodiments, the concentration of beta hydroxy acid may be from about 0.3% (w/w) to about 2.2% (w/w).

In some embodiments, an anti-oxidant composition may have any suitable form for topical application. For example, an anti-oxidant composition may be formulated as a liquid, a cream, a paste, a gel, a jelly, a peel, an ointment, a rub, and/or a powder. An anti-oxidant composition may be applied, according to some embodiments, by wiping, rubbing, smearing, spraying, coating, or otherwise contacting the material with the skin of a subject.

### Devices and Systems

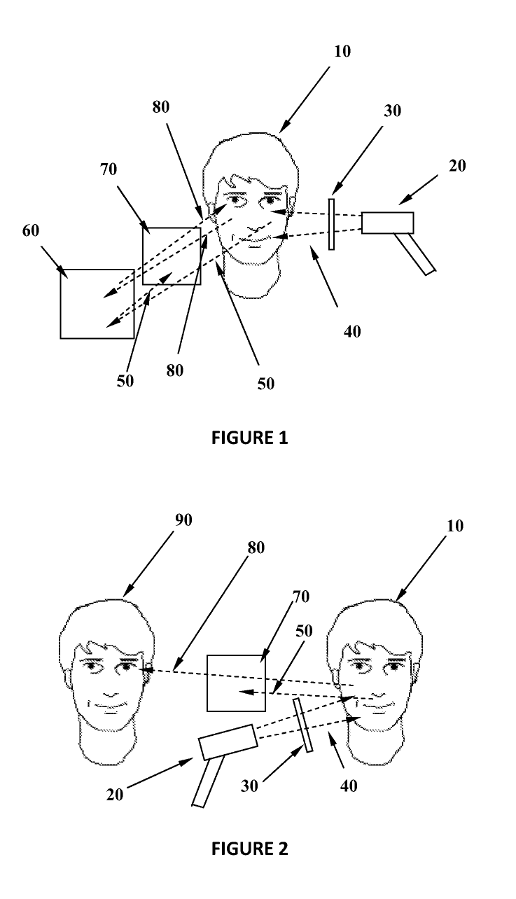

An embodiment of a system in accordance with the present disclosure is shown in FIG. 1. A patient 10 is illuminated with light from light source 20. A light source 20 may comprise light emitting diodes, laser diodes, flashlamps, or other light sources emitting light in the wavelength range of about 390 to about 430 nm, to overlap with the optical absorption in the Soret bands of porphyrins produced by P. acnes bacteria. P. acnes porphyrins may also be excited at other absorption bands such as the Q-bands having various absorption peaks in the range 550 nm to 700 nm. Light in the 600-700 nm range may also induce an anti-inflammatory effect in tissue, although the anti-inflammatory mechanism in this wavelength range more probably also involves the mitochondria. Therefore, in some embodiments, a light source may also encompass these longer wavelengths in the 600-700 nm range, either by a source with a broader spectral range, or by a source comprising multiple LED's or laser diodes operating at different wavelengths. These longer wavelengths may have the advantage of penetrating deeper into the skin than shorter wavelengths.

In some embodiments an optical filter 30 is interposed between the patient and the light source to reduce and/or eliminate undesirable wavelengths from light 40 that illuminates the patient. For example, light emitted from LED's may contain undesirable light in wavelength bands other than the dominant wavelength of the LED. This undesirable light, although of low relative intensity, may hinder observation of the fluorescence due to the low intensity of the fluorescence emission itself. For example, filter 30 may be configured to prevent the patient's skin from being illuminated with light of the same wavelength as that at which the porphyrins in the P. acnes bacteria fluoresce. An example of such a short-pass filter is a model BG3 from Schott North America, of Elmsford, N.Y. In order to reduce specular reflection from the skin, filter 30 may, in some embodiments, be a polarizing optic.

Another means for reducing emission of light from the LED at undesirable wavelengths is to remove those portions of the LED which may be the source of the undesirable emission. Such LED's are available from Medical Lighting Solutions, Inc. of Jacksonville, Fla. This may, in some embodiments, reduce or obviate the need for filter 30.

Light 50 remitted from the patient's skin comprises a portion of light 40 from light source 20, together with fluorescence 80 from the porphyrins in the P. acnes bacteria. Depending upon whether the system is configured for observation of the affected areas, a second optical filter 70 is provided in at least some embodiments to block remitted light from source 20, so that only fluorescence 80 reaches the observer. However, filter 70 is not needed in all embodiments. In some embodiments, optical filter 70 is provided in the form of glasses such as, for example, the model 700-ARG manufactured by the NoIR Laser Company, LLC, of South Lyon, Mich. In some embodiments, a device may include mirror 60, which may be configured to permit a subject to observe affected areas, indicated by areas of fluorescence (e.g., for self-treatment). In addition to and/or instead of mirror 60, a device and/or system may include a camera, a photodetector or visualization means.

With a subject's skin illuminated in the affected areas, typically the face, chest, shoulders, or back, the subject (e.g., a patient or an observer) may visualize the intensity and location of the fluorescent bacteria. This permits the treatment process to be localized to only the affected areas. In some embodiments, optical filter 70, which again may be a pair of glasses, may transmit light in the range of 550 to 700 nm, to allow for a variety of porphyrins with different fluorescence spectra to be observed. In some embodiments, filter 70 simply blocks light below approximately 550 nm. According to some embodiments, light source 20 may be configured to emit light across a broad range of wavelengths and/or in multiple ranges of wavelengths. In such arrangements, optical filter 70 may be configured to filter out some or all of the ranges emitted by source 20.

According to some embodiments, a treatment regime may comprise (e.g., begin with) identifying affected areas in need of treatment (e.g., skin containing active acne lesions), for example, by visualizing fluorescence. It may be desirable, in some embodiments, to proceed without identifying affected areas. For example, a user may treat regions of the skin containing active acne lesions, or may treat prophylactically regions of the skin that may not contain active acne lesions.

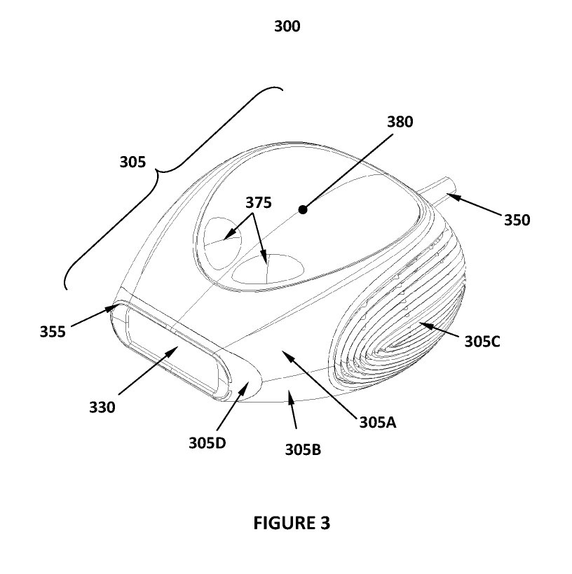

In some embodiments, a treatment device may be configured to be actuated to illuminate the affected areas with an appropriate dose of light at a predetermined wavelength. FIG. 2 illustrates an example embodiment of a visualization device in accordance with the present disclosure. For clarity, elements that are the same as in FIG. 1 are assigned that same reference numerals. Subject 10 is illuminated by light source 20 with light 40 of an appropriate wavelength, such as 413 nm, typically although not necessarily through a filter 30. Light 50 reflected or remitted by the skin of the subject is filtered out by filter 70, while fluorescence 80 passes through filter 70 and may be observed by a physician or other observer 90.

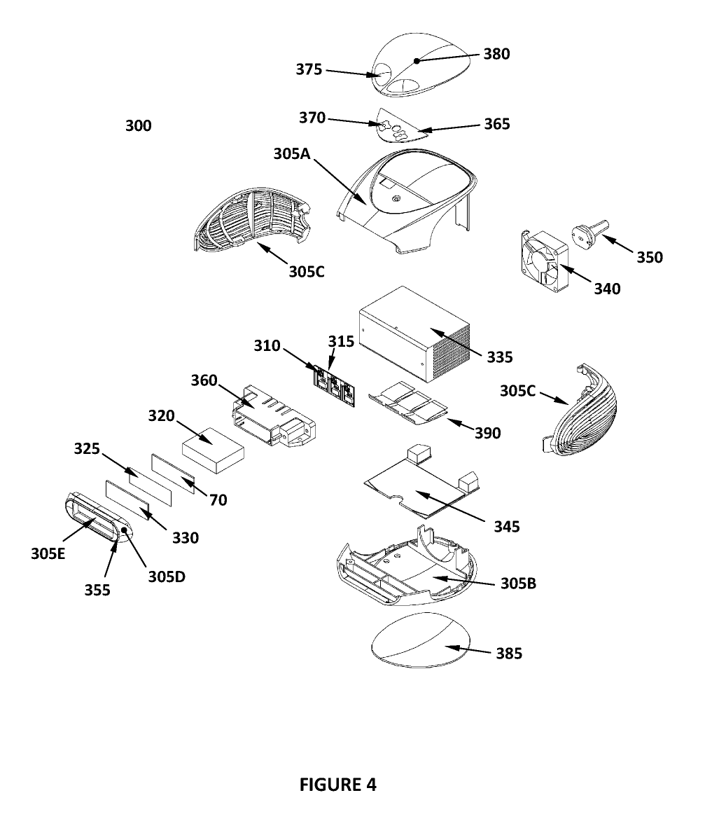

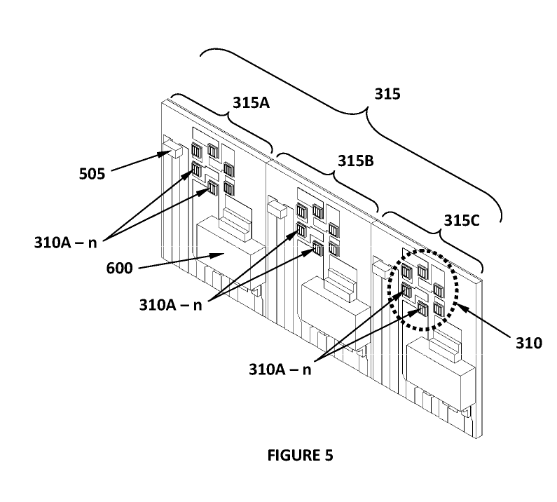

FIG. 3 and FIG. 4 illustrate a specific example embodiment of a treatment device in accordance with the disclosure may be better appreciated. In particular, device 300, shown in exploded perspective view in FIG. 4, comprises a housing 305 which in some embodiments, may be comprised of a top housing 305A, a bottom housing 305B, a vent 305C and a nosepiece 305D, which provides an output aperture 305E. For the illustrated embodiment, the housing is configured to be hand held. It will be appreciated that other embodiments need not be entirely hand held, but may comprise a base station and hand-held head unit connected by an umbilical, or any other suitable physical arrangement.

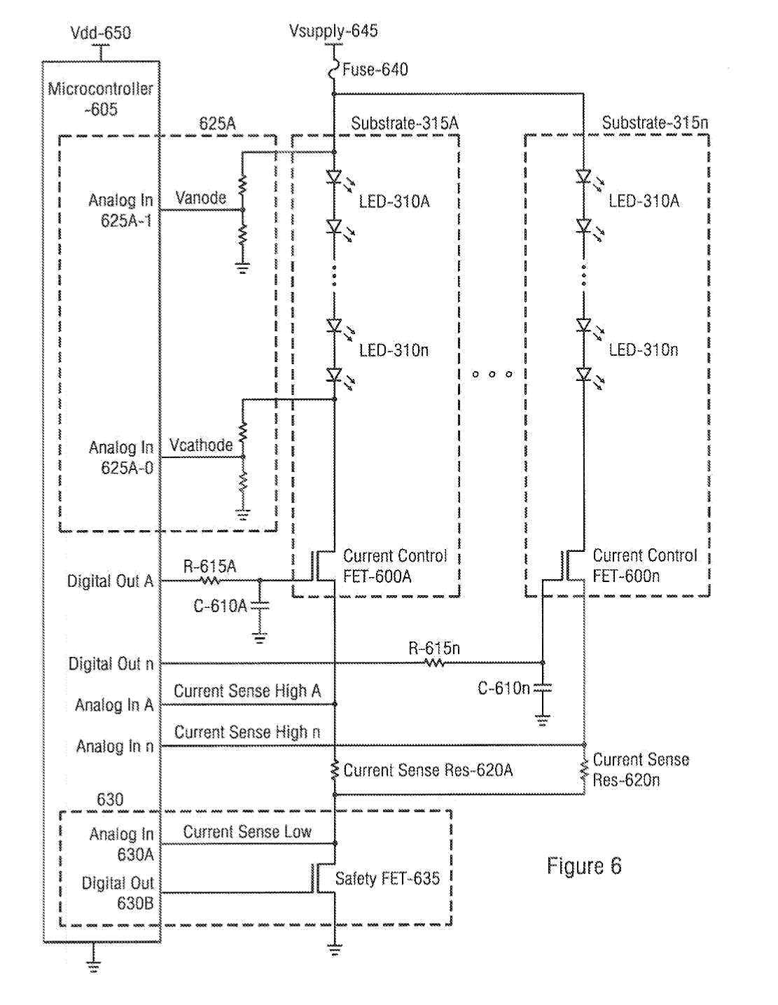

Inside the housing 305 of the illustrated embodiment is a circuit board 315 onto which is mounted a light source 310, which may, for example, be one or more devices such as an LED, an LED array, or other suitable source including one or more laser diodes, flashlamps, or other light emitting devices. In some embodiments, light emitted by source 310 is in the range of 380-500 nm, and in some embodiments is in the range 400-420 nm, such as for example, 413 nm and/or 415 nm. Blue light of 415 nm at a dose of 300 to 500 Joules/cm² over the face may be sufficient to reduce or alter P. acnes colonies to achieve a reduction in acne lesion counts by over 50%. Under some conditions, it has been observed by the present inventors that a power density of 200 mW/cm² is insufficient to produce this effect in less than 4 weeks. Under some conditions, a power density of 800 mW/cm² may induce deleterious side effects. In some embodiments, a desirable power density may be 300 to 700 mW/cm². The size of light source 310 may be determined by aperture size and desired output power density. In some embodiments, higher output power density is currently believed to result in disproportionately higher treatment efficacy at least up to the limit of patient comfort. Light source 310 and circuit board 315 are illustrated in greater detail in FIG. 5, discussed hereinafter.

In the illustrated embodiment, light emitted by light source 310 passes through an optional optical mixer 320 and then through a diffuser 325 in order to optimize eye safety with respect to maximum permissible exposure (MPE) time for a given optical power. For some embodiments, optical filter 70 may be located within a housing, typically in line with diffuser 325. The forward propagating light then passes through an output window 330. Output window 330 may be glass, sapphire or other similar material such as quartz, diamond, and so on. In addition, window 330 may be coated with a transparent anti-microbial layer such as TiO₂. It will be appreciated that not all of the foregoing elements are required in every embodiment and in some embodiments none of these elements is required.

An output window may be configured in a variety of shapes, including square, rectangular, circular and oval. However, in at least some embodiments, the shape of an output window is rectangular, and may have a short axis on the order of one centimeter and a long axis on the order of two to five centimeters. In some embodiments, an output window is rectangular and on the order of one centimeter by three centimeters, which appears to provide a good combination of subject comfort and speed of treatment while also allowing ease of positioning on a subject's face.

Optical mixer 320 may be comprised of a suitable transparent material such as polymethyl methacrylate (acrylic), or glass (BK7 or similar), or quartz. Optical mixer 320 may also be a hollow tube with reflective walls. A diffuser may be a bulk diffuser such as opalized glass, Teflon, or similar scattering media. Diffuser 325 may, in some embodiments, also be a surface scatterer such as ground glass, or engineered substrates having surfaces composed of a multiplicity of microscopic diffractive or refractive elements as for example may be fabricated by lithographic, holographic or other means. Even with sources such as LED's which have a nearly Lambertian output distribution, the eye-safety of a light source is optimized in some embodiments by the use of a diffuser to create a nearly Lambertian virtual source at the output plane of a diffuser with a larger area than the sum of the output area of the individual LED's.

The housing in the illustrated embodiment also contains a heat sink 335, to which circuit board 315 may be mounted. A fan 340 may also be mounted within housing 305 in the event additional cooling is deemed desirable. A fan 340 may be provided to supplement heat sink 335. Alternatively, fan 340 may instead be a blower or similar device for achieving forced convection. Heat sink 335 may have fins that are splayed so that the resistance to airflow is reduced with respect to a heat sink with a similar front surface having fins that are not splayed. A thermo-electric cooling device may also be used in some embodiments either in the alternative or in addition to the heat sink and fan.

A second circuit board 345, also contained within housing 305, provides mounting for a microcontroller and other low-power components not requiring low thermal impedance to the ambient. Power to a device may be supplied by means of a battery (not shown) or connection via conductor 350 to an electrical mains or an external supply. Circuit boards 315 and 345 may be connected by any suitable means, such as a ribbon cable or flexible circuit board 390, for example, one comprised of polyimide substrate so that it may withstand the high assembly temperatures that may be used to affix components to circuit board 315. In at least some embodiments, a rechargeable battery may be used, which may, for example, be nickel-metal hydride, lithium ion, lithium ferrous phosphate, or other rechargeable design.

In some embodiments, skin sensors 355 are also positioned on nosepiece 305D, and may also, for example, be positioned on either side of an optical chassis 360. Sensors 355 may be either capacitive, as disclosed in U.S. patent application Ser. No. 12/189,079, incorporated herein by reference, or may be mechanical or optical, and are intended to ensure close proximity or contact with an area undergoing treatment. Optical chassis 360 supports mixer 320, filter 70, diffuser 325 and output window 330 in at least some embodiments, although some of these components may alternatively be supported by nosepiece 305D. In addition, an on-off switch may also be enclosed within the housing, together with one or a plurality of capacitive sensors 355, which may be positioned around output window 330 in some embodiments.

Also contained within housing 305, in some embodiments, is a board 365 supporting switches 370. Although only two switches are shown, the exact number is determined only by the particular implementation, and may be one or more. In the illustrated example, switches 370 are actuated by buttons 375 positioned on top grip 380. Depending upon the embodiment, switches may be used to turn on power to the device, and/or to cause light source 310 to emit light. In some embodiments, the function of switches 375 may be performed by sensors 355 as discussed above. On-off switch(es) 370 and/or sensors 355 are connected to circuit board 345, directly or indirectly. For convenience, a bottom grip 385 may also be provided, and may be affixed to housing bottom 305B by any convenient means.

FIG. 5 illustrates a specific example embodiment of a device including thermally conductive circuit board 315 and light source 310. Circuit board 315 may be configured of ceramic, such as BeO or AlN, or diamond, or any other material suitable for a thermal environment of a device of the present disclosure. In general, circuit board 315 should be thermally conductive while being electrically non-conductive. In the illustrated embodiment, circuit board 315 is comprised of three substrates 315A-C, but any convenient number of substrates may be used. One or more light sources may be mounted onto each substrate of circuit board 315 in various convenient arrangements, such as the illustrated array of six LED's on each of three substrates. As noted above, the number of sources is largely determined by the desired aperture size and output power density. LED's with emission at a suitable wavelength and power are available from several sources, including, Medical Lighting Solutions, Inc. of Jacksonville, Fla., Cree, Inc. of Durham, N.C., or Nichia Corporation of Tokyo, Japan.

In addition, in at least some embodiments, a temperature sensor 505 such as a thermistor or semiconductor-based thermal detector is also mounted on circuit board 315 to prevent overheating. Additionally, any high power electronics, such as current control FET 600, that would benefit from low thermal impedance to the ambient may be assembled onto circuit board 315. A circuit board 315 that is both electrically insulating and thermally conductive that comprises the LED's, temperature sensors, and high power electronics permits circuit board 345 to be designed with neither extraordinary provisions for heat dissipation nor a means for separately detecting the heat sink/LED array temperature.

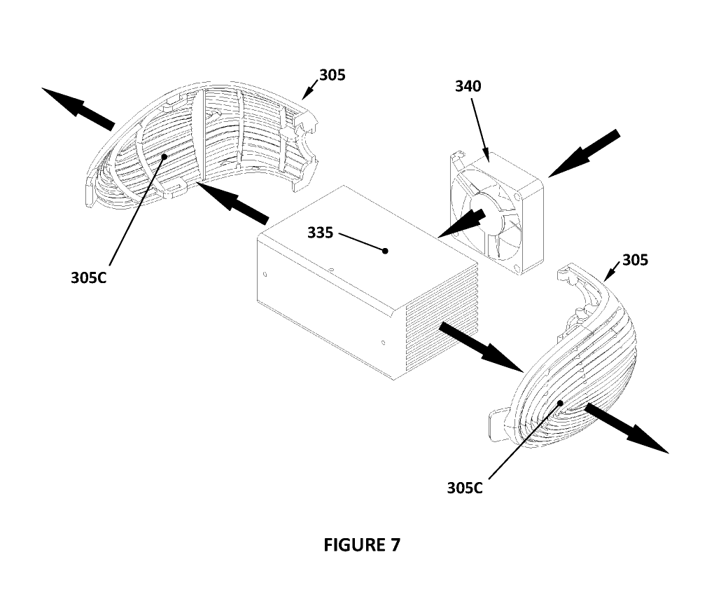

FIG. 6 illustrates a specific example embodiment of control circuitry of the embodiment shown in FIG. 3 and FIG. 4. Drive electronics for the high power light source 310 may include buck, boost, or buck-boost architectures. These architectures employ the use of relatively high-energy inductors to control current for the LED's. According to some embodiments of the treatment device of FIG. 3 and FIG. 4, LED current to the one or more LED's 310A-n on each substrate 315A-n may be controlled using a single FET 600 (shown as FET's 600A-n for n substrates) operating in a linear mode (FIG. 6). Current control FET 600 may be located remotely on the same ceramic substrate 315 on which one or more LED's 310 are mounted in order to take advantage of the low thermal impedance of such a configuration. The remaining circuitry components do not dissipate excessive heat so they do not require any special thermal consideration and may be assembled onto a conventional FR4 printed circuit board 345.

Simple and inexpensive microcontrollers 605 often do not have facilities to provide analog outputs suitable to drive the gate of current control FET 600. In some embodiments, a simple digital output from microcontroller 605 using pulsewidth modulation, together with a single capacitor 610 and a resistor 615 as a low pass filter, may be used to generate a suitable quasi-DC control signal to drive the gate of each FET 600. Thus, in the illustrated embodiment, for n substrates, capacitors 610A-n and resistors 615A-n are used, although this arrangement is not required in all embodiments. A current sense resistor, shown as 620A-n, in series with the LED's may be used to provide feedback to the microcontroller for proper current set point. It may be seen from the circuit diagram in FIG. 6 that this circuit architecture also permits use of common low-voltage microcontrollers powered by voltage supply Vdd 650, that may provide a separate, distinct voltage as that provided by voltage supply Vsupply 645. Voltage supply, Vsupply 645, provides a voltage greater than the sum of the forward voltage(s) of the LED(s) comprising high power light source 310. A voltage required to overcome the forward voltage of more than a few series LED's would damage common, low voltage microcontrollers. Since only two pins of a microcontroller are required to interface and control the high power light source, the use of especially small and inexpensive microcontrollers is possible. Even so, sophisticated functionality such as multiple optical output power settings, slow turn-on and turn-off, and dimming are possible. This simple and inexpensive architecture may achieve electrical efficiency similar to more complicated buck-boost architectures through careful selection of the value of Vsupply so that only a small voltage is dropped across current control FET 600.

In some embodiments, the circuitry shown at 625A provides, for each array of LED's 310A-n, voltage dividers that enable the microcontroller to sense the forward voltage of the LED array so that a non-functional, shorted LED may be detected. It is desirable in some embodiments to detect a shorted LED because the optical output power would decrease and result in diminished treatment efficacy. Also, the forward voltage of one or more shorted LEDs would appear across current control FET 600. The additional voltage across FET 600 would cause additional heat to be generated and could lead to failure of the FET if the microcontroller were to continue to operate the device. Fuse 640 provides an additional safety measure. It will be appreciated that, while only circuit 625A is shown in FIG. 6, similar sense circuits are implemented in at least some embodiments, such that sense circuits 625A-m actually exist.

One skilled in the art of electronics may appreciate that the circuitry discussed to this point may be appropriately duplicated so as to independently control, in parallel, multiple LEDs or multiple LED arrays using multiple control FETs on one or more LED array assemblies 310. The additional components needed are a few resistors and a single capacitor—all low power and inexpensive. Each parallel array requires the availability of a modest number of additional microprocessor pins. The additional, parallel LED arrays may be of the same wavelength or provide for distinct optical wavelengths within the same device.

Safety circuitry 630 shows an additional safety FET 635 that may be used as a backup to current control FET 600 in some embodiments, together with to a current sense low line tied to an analog input of controller 605, and a digital out signal 630B tied to the gate of safety FET 635. Intended to merely act as a switch and not to control the level of current flowing through the LED array, FET 635 may be a small inexpensive FET that does not need to dissipate the large amounts of heat dissipated by current control FET 600. If the voltage dropped across FET 635 is significant compared with the voltage appearing across the current sense resistor, then an additional current sense input to measure the voltage of the negative terminal of current sense resistor 620 may be used. Safety FET 635 may be used to stop current flow to the LED array(s) in the event current control FET 600 fails. In addition to its function as a safety device, and since the gate of safety FET 635 is driven directly by a digital output of the processor and has no interposed RC filter, the safety FET 635 provides the ability to modulate the light source current at higher frequencies than is possible with current control FET 600. By modulating safety FET 635, it is possible to precisely dim the light source to especially low average optical power without the need to resolve the very low current levels required if a DC current level were used to drive the light source. Only one safety FET 635 is required even for multiple parallel LED array assemblies, although additional such FET's may be used if desired.

During operation, device 300 as illustrated in FIG. 3 and FIG. 4 is placed against, or at least near to, the affected area. Sensor(s) 355 or switch(es) 370 trigger the energizing of the LED array, promptly after which a pulse or continuous beam is emitted at a wavelength of approximately 413 nm. In some embodiments, a device may emit a beam with power density of approximately 1 W/cm² and the affected area of the skin may be illuminated for 15-30 seconds. In some embodiments, it may be desirable to significantly increase the power density, for example to 2 W/cm² or, in some embodiments, as much as 10 W/cm² or more. In such arrangements, it may be desirable to cool the skin, either through the use of a coolant mechanism such as a cryogenic spray onto the area for treatment, or to use a thermally conductive window, such as sapphire or the like, and maintain contact between the thermally conductive window and the skin being treated. The window may also be cooled in some embodiments.

Due to the visualization process described in connection with FIG. 1 and FIG. 2, a treatment device may be targeted to the affected areas. The power density of the device may be in the range of 0.5 to 2 W/cm², where a power density of about 1 W/cm² appears to offer, for Caucasian skin, a good compromise among comfort, treatment speed and electrical/optical design considerations where the treatment mechanism is a combination of photochemical and photothermal effects. By cooling or heatsinking the skin, a good compromise among comfort, treatment speed and electrical/optical design considerations may be achieved at power densities of up to 20 W/cm² or higher. In addition, a dose on the order of 20-40 Joules/cm² has been found to be effective for reducing lesion counts. However, it will be appreciated that equally therapeutic effects may be achieved by different doses depending on power density, pulse duration, treatment frequency, treatment interval, and possibly skin type, and therefore the foregoing dosage range and related parameters are not intended to be limiting. Further, in some embodiments, it is desirable to provide a heat source for heating the skin as an additional treatment mechanism, in addition to the treatment techniques described above.

At the lower end of the foregoing dosage range, the treatment mechanism is largely based on the photochemical reaction of light with the porphyrins contained within or proximal to the P. acnes bacteria. At higher dosages, for example those well in excess of 1 W/cm² the treatment mechanism may be primarily photothermal, in which the thermal trauma to the bacteria is believed sufficient to break the inflammation cascade, although photochemical mechanisms may still be involved. One mechanism by which photothermal treatment may be effective is lysing of the bacterial apoptotic vesicle. It will be appreciated that embodiments of the present disclosure may be implemented which use either or both treatment mechanisms, and accordingly different dosage ranges.

Determining the optimum dosage may also involve aspects of eye safety. Diffuser 325 is provided primarily for the purpose of increasing, up to its optimum in some embodiments, the maximum permissible exposure (MPE) of the device, as MPE is defined by the International Standard for the photobiological safety of lamps and lamp systems, (IEC 62471). Other standards may also exist and provide similar guidance. Unlike the photothermal injury associated with some devices, such as those for hair removal, the issue of eye safety in the wavelength range of the present disclosure also involves a photo-chemical reaction in the retina of the eye, which tends to be more restrictive than the photothermal limit at these wavelengths. To prevent damage to the eye, a limit on the amount of exposure per day may be imposed. Such an exposure limit may be implemented by a timer integrated into the electronics of the device that would allow the device to be active for only a limited time per day. One example of a suitable diffuser is a 0.003″ thick wafer of Teflon PTFE 7A, manufactured by DuPont Fluoroproducts, Inc. of Wilmington, Del.

Photon recycling may also be helpful in the device of the present disclosure. If the mixer has side walls perpendicular to the plane of its input and output faces, and the index of refraction is greater than ˜1.41, then no light will escape the mixer through its side walls because all rays incident on the side walls will experience total internal reflection (TIR). Thus, if the source is substantially reflective, any light returned to the source is again reflected back to the diffuser. The mixer serves to spatially homogenize the light so that, at the diffuser of the device, the intensity of the beam is spatially uniform, thus avoiding hot spots. A mixer which ideally has flat side walls and thus cross-sections that are polygonal, such as square, hexagonal, etc., will achieve a high degree of spatial uniformity. Mixers with curved side-walls do not tend to achieve spatial uniformity in all cases but may be useful in some embodiments. Other shapes may be used in other embodiments.

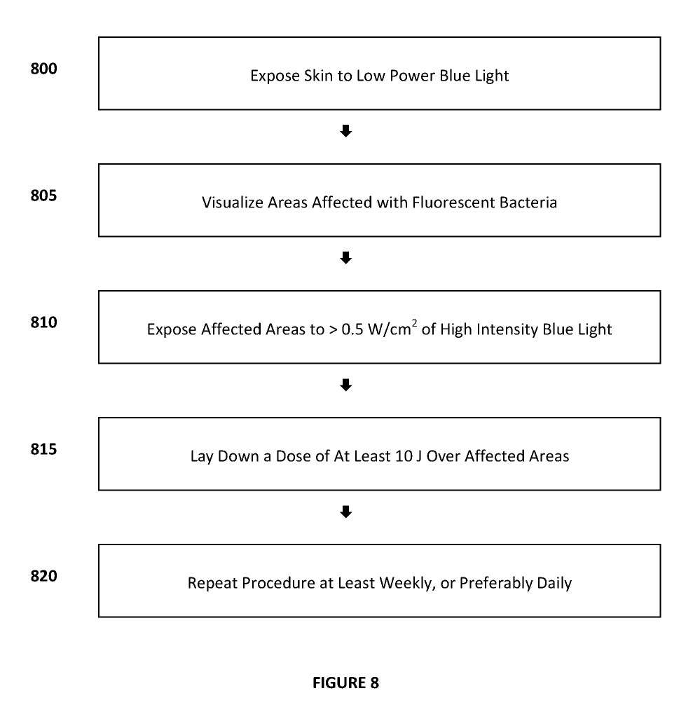

With reference next to FIG. 7, the airflow of the present device may be better appreciated. As shown in FIG. 4, a fan 340 is provided and placed behind heat sink 335. In some embodiments, fan 340 draws air into the device through an inlet in housing 305, where the air is forced past the fins of the heatsink and then out the vent portion of housing 305C. As noted above, alternative heat-management arrangements include a blower, or one or more thermo-electric devices may be used.

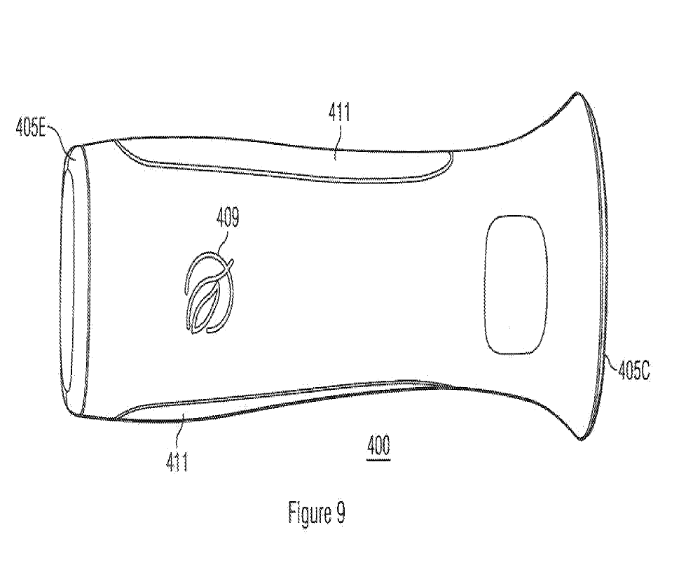

Referring next to FIG. 8, the process for use of the present disclosure may be better appreciated. As shown at step 800, the process begins by illuminating the skin of a subject with low power light of a wavelength that will cause the porphyrins produced by the P. acnes bacteria to fluoresce, either from optical absorption in the Soret band or one or more of the Q bands. Absorption of the Soret and one or more Q bands peak at various wavelengths. Because penetration depth varies with wavelength, light composed of select wavelengths matched to the absorption of the Soret and various Q bands may be employed to optimize the treatment of tissue at various depths. Then, as shown at step 805, one may identify or visualize those areas colonized by the fluorescent bacteria. Next, as shown at step 810, expose the affected areas to high intensity blue light at a sufficient power density, for example about 0.4 watt/cm² or greater.

As shown at step 815, the user lays down a dose on the order of at least 10 Joule/cm² over the affected areas. Various methods may be used for application of the desired dose. In some embodiments, the device is used to “paint” the skin by slowly moving the device over the skin while the device continuously emits light. The user may be instructed to move the device slowly while not keeping the device over the same area of skin so long that the skin becomes uncomfortably hot. The sensation of warmth may be relied upon by the user as an indicator to move to an adjacent location of tissue. Alternatively, a timing mechanism may be provided to indicate when to move the device to the next area of skin, such as an audible beep or buzzer, a visual indicator, a vibration source, or a mechanical roller. Alternatively, the user may be instructed to treat an affected area for a pre-determined about of time per unit area. Another alternative is to monitor the fluorescence quenching achieved by the device, and use that feedback to indicate to the user when to move to the next area. Such a monitor may employ an optical fiber to unobtrusively and conveniently sample the fluorescence emitted by the tissue and convey the light to a suitable detector. In some embodiments, a pulsed device is used and the device is touched to the skin briefly for a single treatment pulse, then lifted and moved to the next treatment area. This approach may be thought of as the “stamping” approach. Such pulsed operation is particularly suited to devices capable of generating 5-20 W/cm² with pulses only a fraction of a second to several seconds in duration.

Finally, as shown at step 820, the user may repeat the process on a regular basis, such as daily or weekly, initially to reduce the lesions and then to maintain the concentration of P. acnes bacteria at a sufficiently low level to reduce their ability to induce further lesions.

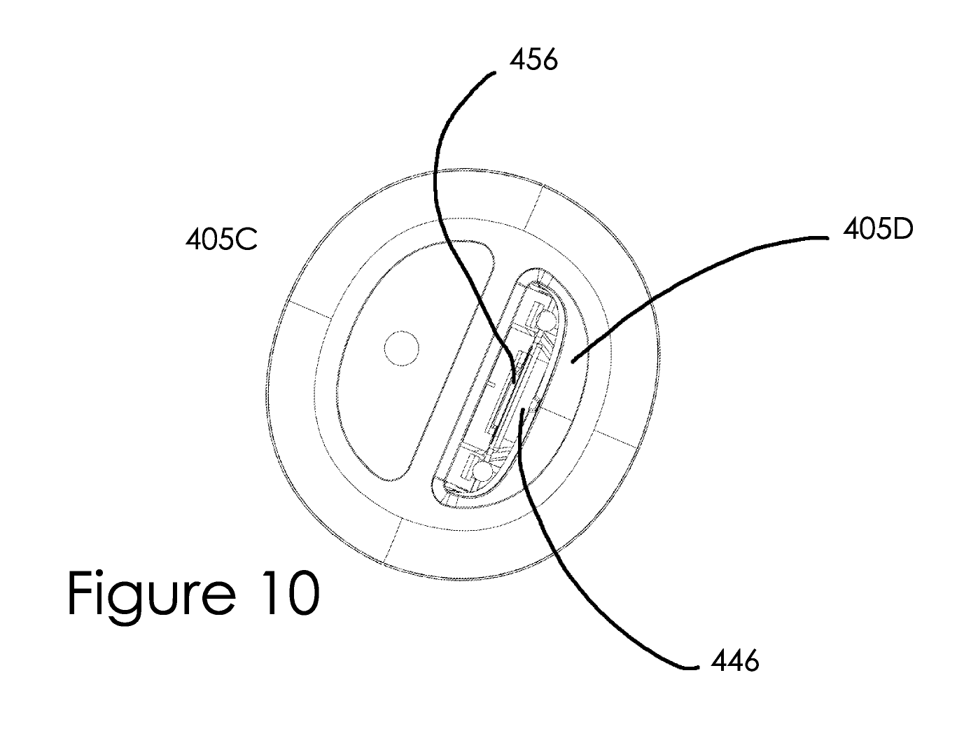

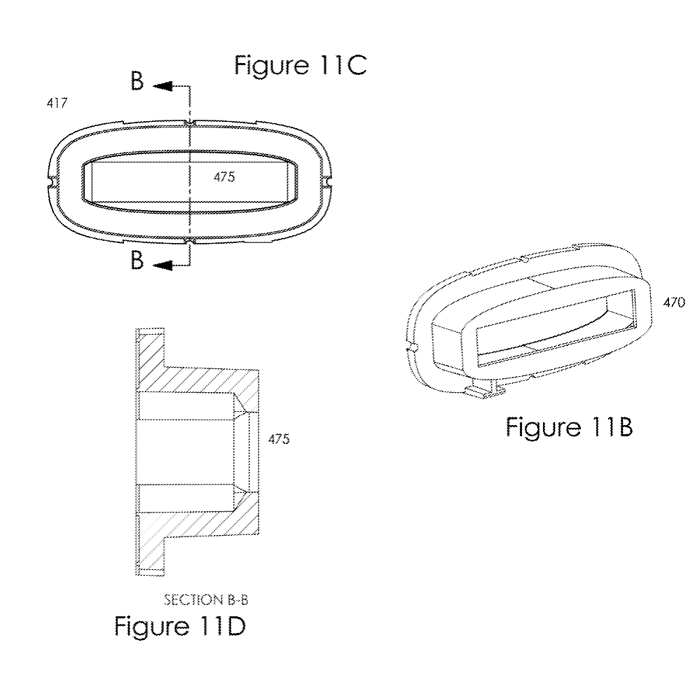

Referring now to FIGS. 9 through 11, an exemplary second embodiment of a treatment device in accordance with the invention can be better appreciated. In particular, the device 400, shown in exploded perspective view in FIG. 11, comprises a housing 405, which is comprised of an upper housing 405A, a lower housing 405B, cap 405C, which provides cap aperture 405D, and a nosepiece 405E, which provides an output aperture 405F. Suitable materials for the housing 400 include, but are not limited to, polymers and polymer blends, such as a polycarbonate/ABS (acrylonitrile butadiene styrene) blend, and it will be recognized by those skilled in the art that other materials, such as light-weight metals and other plastics can also be utilized for the housing. In the illustrated embodiment, the bezel or front of the nosepiece 405E is made of nonconductive material such as plastic, although in other embodiments the nosepiece 405E can be made of metal or metalized plastic.

Although treatment device 400 is battery powered, alternatively, the device can be attached to an external power source using external power conductor 406 which is mounted with screws to the housing 405 and communicates with housing external power aperture 407. The housing 405 can include a decorative design or logo 409, and in the illustrated embodiment, the design element is a cut-out logo design in the housing and can be backlit by light 408 installed within the housing 405.

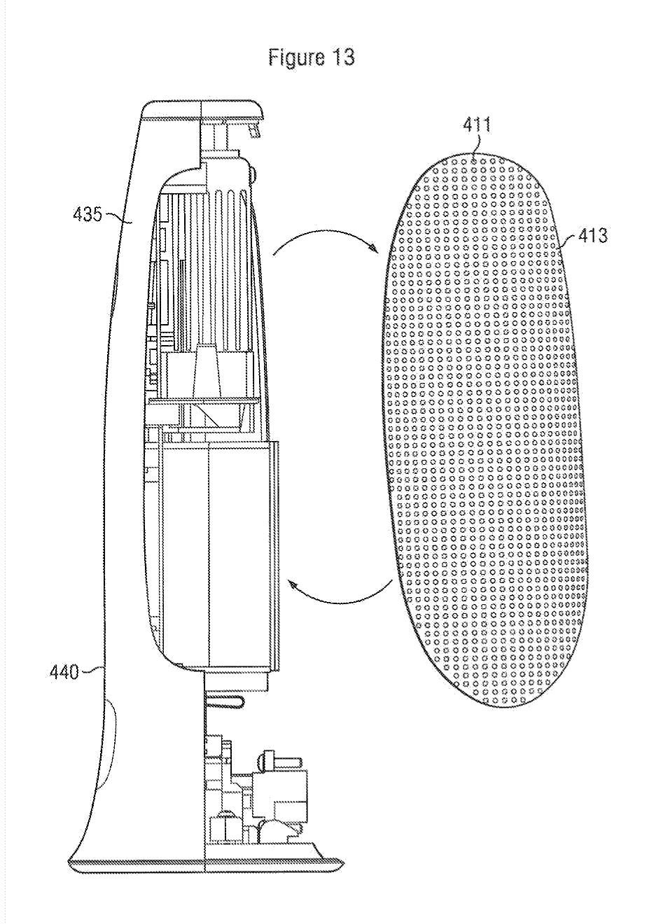

A vent 411 made of a lightweight material such as aluminum is disposed on each side of treatment device 400. The aluminum material of the vents 411 is configured as a mesh having multiple apertures, and each vent 411 includes both air intake and air outlet regions, as described more fully below in connection with FIG. 13.

In the illustrated embodiment, the housing 405 is configured to be hand held and is generally shaped as a tapering, somewhat flattened cylinder. It will be appreciated that other embodiments need not be entirely hand held, but can comprise a base station and hand-held head unit connected by an umbilical, or any other suitable physical arrangement.

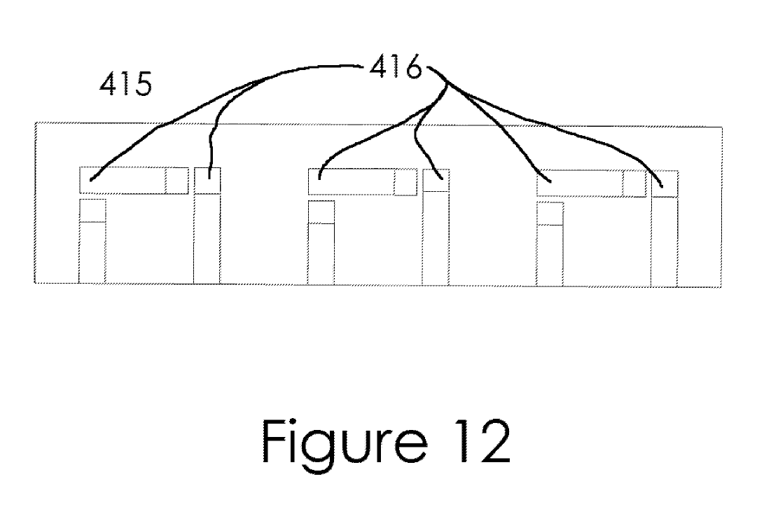

Inside the housing 405 of the illustrated embodiment is a circuit board 415 onto which is mounted a light source 416, which can, for example, be one or more devices such as an LED, an LED array, or other suitable source including one or more laser diodes, flashlamps, or other light emitting devices. In at least some embodiments, the light emitted by the source 416 is in the range of 380-500 nm, and in an embodiment is in the range 400-420 nm, such as for example, 413 nm. The size of the light source 416 is determined by aperture size and desired output power density. In this exemplary embodiment, the light source 416 is six or eight LED's mounted on a single BeO ceramic circuit board 415, which can also be made from, for example, AlN, or diamond, or any other material suitable for the thermal environment of the device of the present invention. The light source 416 and the circuit board 415 are illustrated in greater detail in FIG. 12, discussed hereinafter. As noted previously, other embodiments can comprise as few as one suitably powerful LED or as many as twenty or more LED's.

In the illustrated embodiment, light emitted by the light source 416 passes through a hollow optical mixer 417, the tubular wall of which is approximately 1 cm in length. The mixer 417 has reflective walls and is made from aluminum or another light-weight metal, or from metalized plastic. If a solid mixer is preferred for the particular implementation, the mixer can be comprised of a suitable transparent material such as polymethyl methacrylate (acrylic), or glass (BK7 or similar), or quartz. In some embodiments, a hollow mixer is preferred because it allows greater light divergence and thereby enables a more uniform distribution of the light at the outlet aperture 405F.

The mixer 417 serves to spatially homogenize the light so that, at the output side of the diffuser 425, the intensity of the beam is substantially uniform, and hot spots are reduced or avoided. It will be appreciated by those skilled in the art that the term “uniform” as used in this context can still allow for significant variation, depending upon how “uniform” is measured. A mixer which ideally has flat side walls and thus cross-sections that are polygonal, such as square, hexagonal, etc., will achieve a high degree of spatial uniformity. Mixers with curved side-walls tend not to achieve as much spatial uniformity in all cases but can be useful in some embodiments. Other shapes can be used in other embodiments.

The hollow mixer 417 includes a gasket 418, to which a diffuser 425 is attached. The diffuser can be a bulk diffuser such as opalized glass, Teflon, or similar scattering media; in an embodiment, the diffuser can comprise Virgin Electrical Grade Teflon having a thickness of 0.003″ to 0.005″. One such material is Teflon PTFE 7A, manufactured by DuPont Fluoroproducts, Inc. of Wilmington, Del. The diffuser 425 can, in some embodiments, also be a surface scatterer such as ground glass, or engineered substrates having surfaces composed of a multiplicity of microscopic diffractive or refractive elements as for example can be fabricated by lithographic, holographic or other means. From the mixer 417, the light travels through the diffuser 425 in order to optimize eye safety with respect to maximum permissible exposure (MPE) time for a given optical power. The diffuser 425 is provided primarily for the purpose of increasing, up to its optimum in some embodiments, the maximum permissible exposure (MPE) of the device, as MPE is defined by the International Standard for the photobiological safety of lamps and lamp systems, (IEC 62471). Other standards may also exist and provide similar guidance. Unlike the photo-thermal injury associated with some devices, such as those for hair removal, the issue of eye safety in the wavelength range of the present invention also involves a photo-chemical reaction in the retina of the eye, which tends to be more restrictive than the photothermal limit at these wavelengths. Even with sources such as LED's which have a nearly Lambertian output distribution, the eye-safety of the light source is optimized in some embodiments by the use of a diffuser having sufficient scattering characteristics to create a nearly Lambertian virtual source at the output plane of the diffuser while also providing a larger output area for the emitted light than the sum of the output area of the individual LED's.

For some embodiments, an optical filter, such as the filter 325 shown in FIG. 4, can be located within the housing, typically in optical alignment with the diffuser 425. However, such a filter is not required in all embodiments. Ultimately, the forward propagating light passes through the output window 420. Output window 420 is a polycarbonate material, and also can be made of glass, sapphire or other similar material such as quartz, diamond, and so on. In addition, the window 420 can be coated with a transparent anti-microbial layer such as TiO₂.

The output window can be configured in a variety of shapes, including square, rectangular, circular and oval. However, in the illustrated embodiment, the shape of the output window is generally a rounded rectangle, and can have a short axis on the order of one half to one centimeter and a long axis on the order of two to five centimeters. In an embodiment, the output window is a rounded rectangle and on the order of 0.5 centimeter by 3.5 centimeters, which appears to provide a good combination of patient comfort and speed of treatment while also allowing ease of positioning on the patient's face.

A heat sink 435 is provided within the housing 405 and is made of aluminum coated with an adhesive, such as a silver-filled epoxy adhesive, which forms an interface film 436 between the heat sink 435 and the circuit board 415. The heat sink 435 is fixedly mounted within the housing by means of post 438 projecting upwardly from the lower housing, together with screw 437B. A conductor 439 encircles the post 438 and also extends forward to make a good electrical connection with both the underside of the metal-coated mixer 417 and a contact pad (not shown) on the underside of a second printed circuit board assembly (PCBA) 445. A fan assembly 440, mounted to fan mounting bracket 442, is disposed behind the heat sink 435. The fan assembly comprises two fans and is a 1.1 Watt assembly with a voltage of 5.5 VDC, manufactured by Sunonwealth Electric Machine Industry Co., Ltd. The fan assembly 440 is provided to supplement heat sink 435 in embodiments where such supplementation is desired. The fan assembly 440 can be a blower or similar device for achieving forced convection. Heat sink 435 can have fins that are splayed so that the resistance to airflow is reduced with respect to a heatsink with a similar front surface having fins that are not splayed. A thermo-electric cooling device can also be used in some embodiments either in the alternative or in addition to the heat sink and fan.

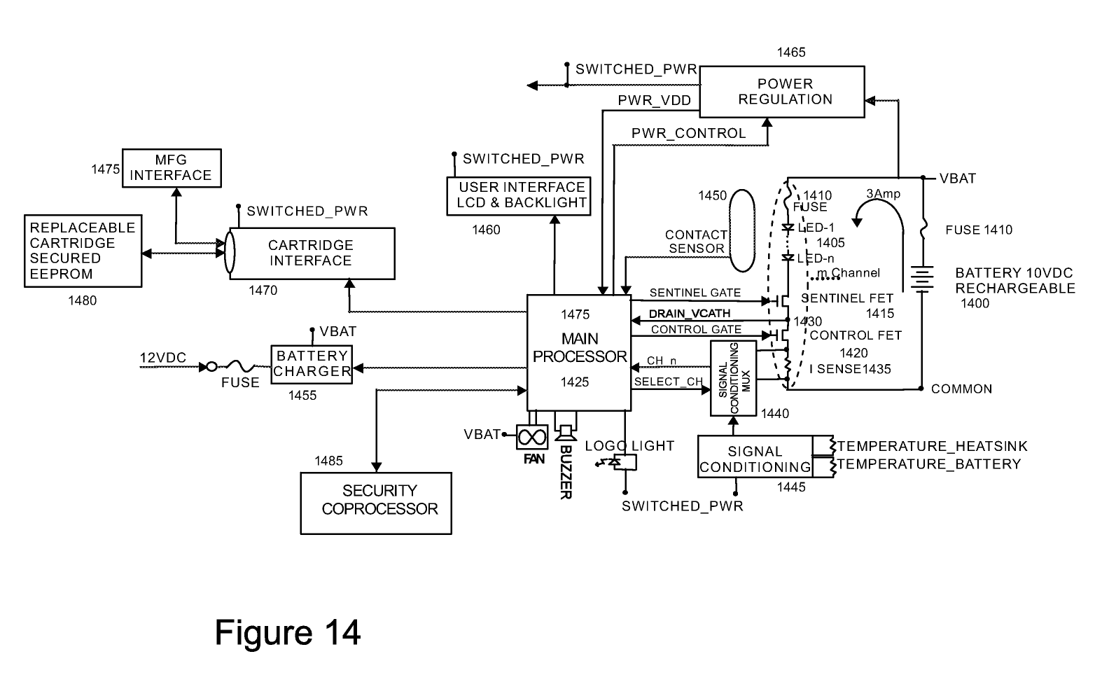

The second PCBA 445, also contained within housing 405, provides mounting for a microcontroller and other low-power components not requiring low thermal impedance to the ambient. The screws 437A provide a good thermal connection between the components on the PCBA 445 and the heatsink 435, and particularly provide a good thermal connection between the heatsink and a control FET, discussed hereinafter in connection with FIG. 14.

In the illustrated arrangement, power to the device is supplied by means of a battery 447, which can comprise, for example, a 3-cell triangular 9.6 VDC battery, although other choices of power sources can be used in other implementations. A poron foam battery support is provided on the top and the bottom of the battery, and both ends of the battery 447 have an insulator layer 449. The device 400 can be connected to an electrical mains or an external supply by conductor 406. The circuit boards 415 and 445 can be connected by any suitable means, such as a ribbon cable 446 or a flexible circuit board 490, for example, one comprised of polyimide substrate so that it can withstand the high assembly temperatures that can be used to affix components to circuit board 415. Foam sheet 446A can be provided to prevent undesirable wear and contact. In at least some embodiments, a rechargeable battery can be used, which can, for example, be nickel-metal hydride, lithium ion, lithium ferrous phosphate, or other rechargeable design.

In this exemplary embodiment, one or more skin sensors 355, as shown in FIG. 4, are also positioned on the nosepiece 405E. The sensors 355 can be either capacitive, as disclosed in U.S. patent application Ser. No. 12/189,079, filed Aug. 8, 2008, incorporated herein by reference, or can be mechanical or optical, and are intended to ensure close proximity or contact between the device and an area undergoing treatment. In some embodiments, the one or more capacitive sensors 355 can be positioned around the output window 330. In others, such as the illustrated embodiment, the mixer 417 can be metal coated and can serve as the capacitive sensor when properly connected to the device's controller, as described above, by means of conductor 439 forming a connection to PCBA 445 and the control electronics mounted thereon. To ensure a good electrical connection through mechanical contact, the conductor 439, which can be copper, for example, can be turned up at the end which contacts mixer 417. Alternatively, in some embodiments the nosepiece 405E can serve as the capacitive sensor, for example when the mixer is a solid mixer, in which case the nosepiece should be made of metal or metalized plastic and connect to the electrode 439. In embodiments where the mixer 417 serves as the capacitive sensor, the nosepiece 405E should not be metal or otherwise electrically conductive, to minimize interference with the operation of the mixer 417 as the sensor.

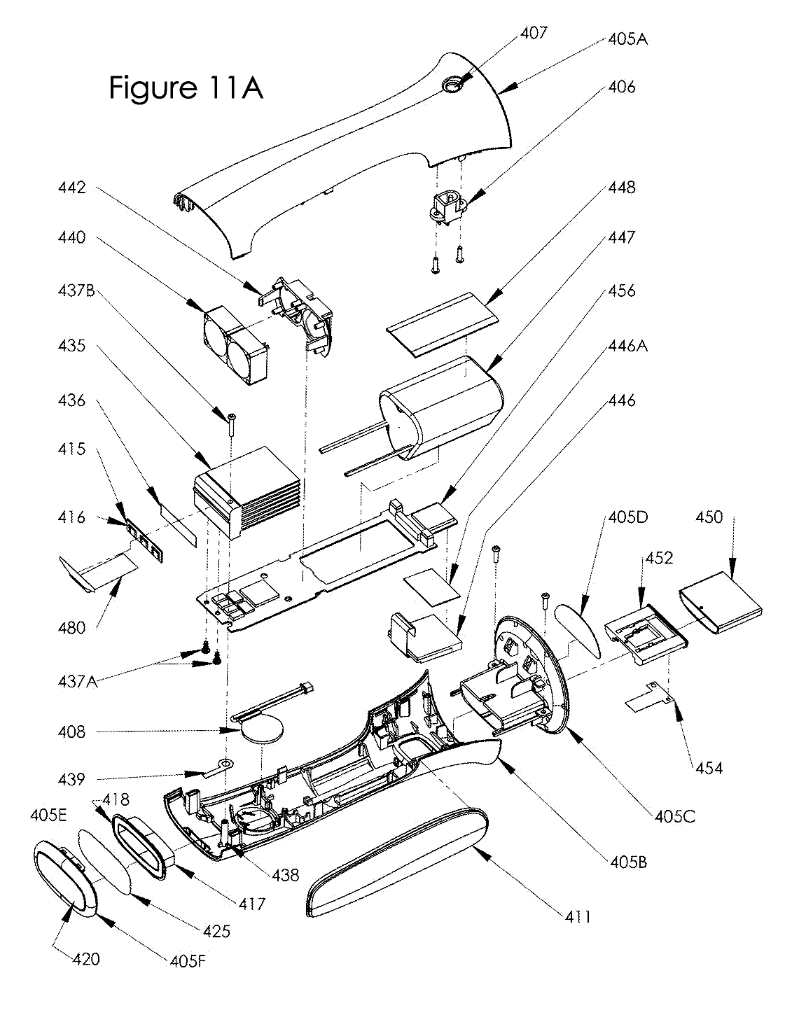

The second embodiment ensures safe and controlled use of the treatment device by the user by controlling activation and timing of treatment through the use the control electronics discussed in connection with FIG. 14. In embodiments which use them, the timing cartridges 450 illustrated in FIG. 14 and in FIG. 4 and FIG. 11 are inserted into the device and can be configured to activate treatment, although in at least some embodiment the sensors 355 discussed above function to turn the device on and off. The cartridges 450 are, in one embodiment, disposable brushed stainless inserts that can be configured to provide different, selectable treatment regimes appropriate for the user. In use, a cartridge 450, which is configured with an electronics regimen a carrier 452 attached to the cartridge 450 by bracket 454, is inserted into the housing through the cap aperture 405D, as best shown in FIG. 10. The inserted cartridge 450 attaches to PCB connector end 456 of the main PCB 445. The selected regime is then executed by the electronics of the PCB 445 to provide treatment. In some embodiments, the cartridge 450 provides a means for storing the amount of time remaining available for use of the device, typically either by recording time of use or decrementing from a pre-stored time value. In embodiments where the cartridge 450 serves to track only the time of use, the control function can be embedded in a controller which forms part of the drive electronics discussed hereinafter. Determining the optimum dosage can also involve aspects of eye safety. To prevent damage to the eye, a limit on the amount of exposure per day can be imposed. Such an exposure limit can be implemented by the timer cartridge 450 that allows the device to be active for only a limited time per day.

Photon recycling can also be helpful in the device of the present invention, although the elements providing the photon recycling differ slightly from those of the first embodiment. In particular, in an embodiment the mixer 417 is hollow, and includes an end wall 470 through which an orifice 475 is formed, as shown in FIGS. 11B-11D. Lights from the LED array enters the mixer through the orifice 475, and the interior of the mixer 417, including the inside portion of the end wall 470, is highly reflective. The diffuser 425 typically transmits approximately 50% of the light illuminating it; the other 50% is returned back into the mixer. That returned light strikes either the LED array or the rear wall, and light hitting the rear wall is returned toward the diffuser. In addition, light transmitted through the diffuser into the skin can also be scattered by the skin and returned to the diffuser. Again, since the diffuser transmits only about 50% of the light striking it, and returns the rest, a portion of the light returned from the skin is re-transmitted back into the skin.

Referring next to FIG. 12, the thermally conductive circuit board 415 and light source 416 can be better appreciated. The circuit board 415 preferably is configured of ceramic, such as BeO or AlN, or diamond, or any other material suitable for the thermal environment of the device of the present invention. In general, circuit board 415 should be thermally conductive while being electrically non-conductive. In the illustrated embodiment, circuit board 415 is a single substrate, and one or more light sources can be mounted onto the substrate of the circuit board 415 in various convenient arrangements, such as the illustrated array of six LED's 416 on the single substrate. In this embodiment, the number of LED's generally is six or eight but can range from a single large LED to twenty or more, as previously discussed. Also as noted above, the number of sources is largely determined by the desired aperture size and output power density. LED's with emission at a suitable wavelength and power are available from several sources, including, Medical Lighting Solutions, Inc. of Oviedo, Fla., Cree, Inc. of Durham, N.C., or Nichia Corporation of Tokyo, Japan.

In addition, in at least some embodiments, a temperature sensor 505 such as a thermistor or semiconductor-based thermal detector as shown in FIG. 5, can also be mounted on circuit board 415 to prevent overheating, although in other embodiments it can be more desirable to mount the temperature sensor 505 on PCBA 445 to ensure a low thermal impedance between the sensor and the heatsink. Additionally, any high power electronics, such as current control FET 600, that would benefit from low thermal impedance to the ambient can be assembled onto circuit board 415. A circuit board 415 that is both electrically insulating and thermally conductive that comprises the LED's, temperature sensors, and high power electronics permits circuit board 445 to be designed with neither extraordinary provisions for heat dissipation nor a means for separately detecting the heatsink/LED array temperature.

Low thermal impedance between the LED junction and the ambient forms an aspect of the present invention, and allows devices built in accordance with this aspect of the invention to drive more electrical current through the die, resulting in greater optical output power, without the creation of more waste heat than can be dissipated without undesirably large increases in junction temperature and without the use of extraordinary cooling efforts. In particular, by use of flip-chip mounted die for the LEDs, which substantially eliminate substrate thermal impedance, together with the use of a Beryllium Oxide (BeO) or similar circuit boards on which to mount the LEDs as well as a suitable heatsink such as the finned aluminum heatsink shown, plus a small boundary layer of air created by forced air convection, a thermal impedance much less that 10° C./Watt can be achieved. In the illustrated embodiment, thermal impedances of approximately 2.7° C./Watt are achieved, whereas conventional LED mounting architectures with package die mounted on a PCB can have a thermal impedance of more than 100° C./Watt, and perhaps as high as several hundred ° C./Watt. This significant reduction in thermal impedance allows the use of fewer LEDs to achieve the desired system power.

Referring next to FIG. 13, the airflow of the present device can be better appreciated. As discussed above, a fan assembly 440 is provided and placed behind heat sink 435. In an embodiment, the intake of the fan assembly 440 draws air into the housing through the intake region 412 of the mesh aluminum vents 411, the intake region being positioned contiguous to the fan intake. The fan assembly directs the air into and through the heat sink 435, where the air is forced past the fins of the heatsink and then out of the housing through the outlet region 413 of the vent 411, the outlet region being positioned contiguous to the outlet end of the heat sink 435. As noted above, alternative heat-management arrangements include a blower, or one or more thermo-electric devices can be used.

Referring next to FIG. 14, aspects of the control circuitry of the embodiment shown in FIGS. 9-11 can be better appreciated. A battery 1400 supplies power directly to a plurality of channels, only one of which is shown in FIG. 14 for purposes of clarity. Each channel comprises a plurality of LEDs 1405 marked LED-1 through LED-n through one or more fuses 1410; for example, a device can have three or four channels of two LEDs per channel, for a total six or eight LEDs. In each channel, the LEDs are series connected to a sentinel FET 1415 and a control FET 1420, the gates of which are controlled by a controller or other processor 1425, which can, for example, be a Freescale MC9S08LL64CLH. The controller 1425 applies appropriate voltage to the gate of control FET 1420 to enable drive current to flow to the LEDs 1405. Some controllers, such as the one noted above, cannot output analog voltages and require a D/A converter, which can be a simple RC circuit as shown in FIG. 6 and not repeated here for clarity. The controller 1425 also monitors the status of the node 1430 between the sentinel FET and the control FET. The controller also monitors the status of each channel by means of a sense resistor 1435, which is sensed through a signal conditioning mux 1440. The signal conditioning mux 1440 also receives inputs representative of heat sink temperature and battery temperature, through a second signal conditioning mux 1445. Thus, it can be appreciated that the controller monitors in real time the LED current, voltage and temperature, as well as the battery voltage, charge and temperature. The sentinel FET essentially functions as a safety switch. While the controller 1425 normally maintains the sentinel FET in the “on” state, in the event an error condition occurs for any of the monitored parameters, the controller defaults to turn off the gate to the sentinel FET, thus disabling the device from energizing the LEDs in that channel. The controller can also turn off the control FET in the event of an error condition, in at least some configurations. A FET switch actuated by the controller can also be provided to disconnect the battery charger 1455.

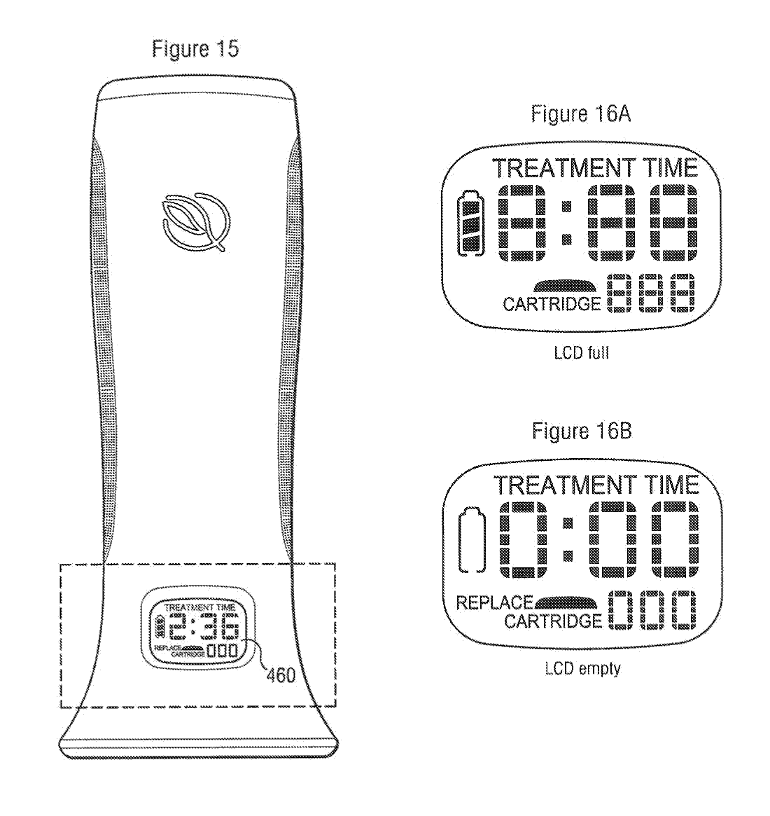

The capacitive or other skin sensor 1450 connects to the controller 1425 through conductor 439 or similar arrangement, as discussed above. The controller provides inputs to the user interface LCD and backlight, indicated at 1460, as discussed hereinafter in greater detail in connection with FIGS. 15 and 16A-B. Power regulation to the controller is provided by regulator 1465 in a conventional manner.

In addition, the controller communicates with a cartridge interface 1470, which serves two functions. During manufacturing, the interface 1470 permits the manufacturing systems to communicate directly with the device through manufacturing interface 1475, thus enabling loading of firmware, system calibration, and testing of system performance. During normal operation, the interface 1470 receives replaceable cartridge 1480, which in some configurations comprises a secure EEPROM that provides to the controller an allotment of treatment time. In other configurations, the cartridge 1480 provides a complete treatment regimen. Alternatively, one or more treatment regimens can be programmed into the controller and its associated memory.

To ensure that the cartridge is authentic and thus does not create an unsafe operating condition, the cartridge 1480 cooperates with the controller and a security coprocessor 1485. The security coprocessor can be a device such as the DS2460 by Maxim, with a corresponding device such as the Maxim DS28CN01 in the cartridge 1480. Authenticity is assured through the use of any convenient security mechanism, such as, for example, a secure hash algorithm. A multi-part authentication scheme can be implemented by storing a first portion of the authentication data in the coprocessor 1485, and a second portion of the authentication in the cartridge. The authentication data maintained in the coprocessor can, in at least some embodiments, be created in the specific unit by means of a sequenced installation process, where the order of the data affects the result, and the full device-side authentication data resides only in the coprocessor. This installation process is managed during manufacturing through the interface 1475 by loading into the device controller “coprocessor initialization” firmware. That firmware places the device in a known and safe state, and then installs at least the first piece of authentication data. In some embodiments, the device is reset after the first piece of authentication data is installed, after which a second piece of coprocessor initialization firmware is loaded into the processor and a second portion of the coprocessor authentication data is loaded into the coprocessor. It can be appreciated that, in some implementations, the authentication data can be loaded in less or more steps that the two described above, with one or more firmware installation functionalities.

In at least some embodiments, the authentication data maintained in the cartridge exists only in each specific cartridge. The authentication data can, in some embodiments, be derived from, for example, all or a portion of the serial number of the cartridge, together with a static portion, plus some or all of the contents of a read-only memory page. Like the main device, the authentication data in the cartridge is installed in multiple steps for at least some embodiments, with the sequence of those steps impacting the final result. In an embodiment, when installed in the device, the cartridge is verified by the coprocessor 1485 through the main controller 1425, and is continually authenticated as long as it is connected to the interface 1470. Once the cartridge is authenticated, the memory in the cartridge is read and the data used by the controller 1425.

During operation, the device 400 as illustrated in FIGS. 9-11 is placed against, or at least near to, the affected area. The capacitive sensor(s) enable the energizing of the LED array, with the timing cartridge 450 controlling the maximum amount of treatment time available, or, in some embodiments, providing the treatment regimen. In such embodiments, the timing cartridge 450 controls emission of a pulse or continuous beam at a wavelength of approximately 413 nm. In an embodiment, the device emits a beam with power density of approximately 0.5 W/cm² and the affected area of the skin is illuminated for 30 seconds.

The power density of the device can be in the range of 0.3 to 1 W/cm², where a power density of less than 0.5 W/cm², and in some instances about 0.3 to 0.4 W/cm², appears to offer, for Caucasian skin, a good compromise among comfort, treatment speed and electrical/optical design considerations. As presently understood, the treatment mechanism is a combination of photochemical and photothermal effects. Such a low dosage further reduces or eliminates hyperpigmentation of the skin following treatment.

Referring next to FIGS. 15, 16A, 16B, and 17, an embodiment of the process for use of the present invention can be better appreciated. The display features shown in FIGS. 15, 16A and 16B provide the user with an indication of the amount of treatment time for a given treatment. As will be appreciated from the following, an embodiment of a treatment regimen includes a prophylactic portion as well as a more intense portion. In addition, the treatment regimen discussed below is divided into a first portion covering the first two weeks, and a second portion covering the period after the first two weeks.

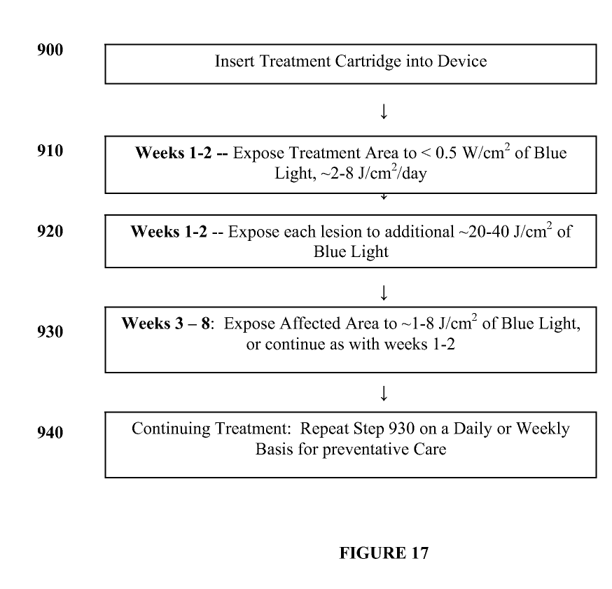

Thus, as shown in FIG. 17, beginning at step 900, the process is enabled by inserting the treatment cartridge 450. Depending upon the embodiment, the cartridge 450 provides an amount of available treatment time, or provides all or part of a treatment regimen. Then, for the embodiment shown in FIG. 17, at step 910, during weeks one and two (days 1-14), the user performs morning and nighttime treatments by illuminating the area of the patient's skin to be treated with light having a power density of about 0.3-0.5 W/cm² and a 413 nm wavelength for three (3) minutes while utilizing a sweeping/painting motion. This results in a prophylactic dose of about one Joule/cm² for each of the morning and night treatments, or a total daily prophylactic dose of about two Joules/cm².

In addition, during each of the morning and night treatments, the user can dwell over lesions for an additional period of approximately 30 seconds each, as shown at step 920, which delivers an additional dose of about 12 Joules/cm² to areas having lesions. Thus, during the first two weeks, each of the night and morning treatments results in a prophylactic dose of about one Joules/cm², and a dwell dosage for areas having lesions of approximately an additional 12 Joules/cm². This results in a daily prophylactic dose of about two Joules/cm² over the treated area and a daily dwell dosage of about 26 Joules/cm² over areas having lesions.

It will be appreciated that, while the embodiment described above contemplates two treatments, other treatment regimens are equally viable and will be apparent to those skilled in the art. The treatment goal is to provide the right daily dosage to the patient, which is typically 1-4 Joules/cm2 as a prophylactic treatment, and 20-40 Joules/cm² for areas having lesions. Thus, one alternative is to treat more times per day, with each treatment being for a shorter time; or, alternatively, a single, longer treatment per day.

Treatment beyond the first few weeks typically eliminates the need for dwelling upon particular lesions. Accordingly, step 930 provides, for instance, the following treatment regime for weeks 3 through 8: The treatment area is treated for 3 minutes with a sweeping/painting motion in the morning and evening, providing an estimated daily dose of approximately two Joules/cm².

As shown at step 940, the treatment regime can be repeated on a regular basis, such as daily or weekly, initially to reduce the lesions and then to ensure that the concentration of P. acnes bacteria remains at a sufficiently low level that the inflammatory cascade is inhibited, and the likelihood that other lesions will form is reduced.

It will also be appreciated that, while steps 930 and 940 are illustrative of one treatment regimen, it is also permissible, and in some cases desired, to continue the regimen of weeks one and two into weeks three and four, and longer if desired. Alternatively, the dwelling portion of the treatments can be omitted, or the prophylactic painting treatment could be reduced in time, for example to two minutes rather than three, or either the evening or the morning session could be omitted.

The device is used to “paint” the skin by slowly moving the device over the skin while the device continuously emits light. The user can be instructed to move the device slowly while not keeping the device over the same area of skin so long that the skin becomes uncomfortably hot. The sensation of warmth can be relied upon by the user as an indicator to move to an adjacent location of tissue. Alternatively, the timing cartridges or the device itself can be programmed to indicate when to move the device to the next area of skin, such as an audible beep or buzzer, a visual indicator, a vibration source, or a mechanical roller. Alternatively, the user can be instructed to treat an affected area for a pre-determined about of time per unit area. Another alternative is to monitor the fluorescence quenching achieved by the device, and use that feedback to indicate to the user when to move to the next area. Such a monitor can employ an optical fiber to unobtrusively and conveniently sample the fluorescence emitted by the tissue and convey the light to a suitable detector.

As shown in FIGS. 15, 16A, and 16B, the polycarbonate treatment device window 460 has a liquid crystal display (LCD) to provide information about the inserted timing cartridge. The LCD display provides, for example, treatment times and an indication of when the cartridge needs to be replaced. It will be recognized by those skilled in the art that the display 460 can also show the amount of power delivered and other parameters of interest, such as a number or name identifying a particular treatment regime.

It will be appreciated that the method, system and apparatus taught herein may effectively reduce the level of colonization of a subject's skin by the P. acnes bacteria. By treating the affected areas as discussed above, the concentration of bacteria in the sebaceous ducts and glands may be significantly reduced. Lower bacterial load reduces the concentration of inflammatory bacterial metabolites, thereby reducing the likelihood of the induction of an inflammatory cascade of the type that produces lesions. Essentially, by breaking the chain of the inflammatory cycle, the present disclosure reduces and prevents the formation of lesions, and/or may enhance the rate at which lesions clear.

Stated differently, some embodiments of the present invention use selective photothermolysis of the pilosebaceous duct, gland and/or contents. It has been determined that the bulk of the material within an infected gland is composed of P. acnes bacteria. This allows selective targeting of absorbing chromophores produced by the bacteria, rather than the sebum produced by the sebaceous gland. This also provides the possibility of delivering a sufficient dose to the affected area within an acceptably short time. The result is a treatment regimen that can also involve reduction of hyperkeritinization, bacterial destruction, and reduction of inflammation. In addition, the ability of the sebaceous gland to prevent leakage of its content into the surrounding dermis can be increased through dietary supplementation of GLA or similar long-chain fatty acids which are typically deficient in the sebum of acne sufferers.

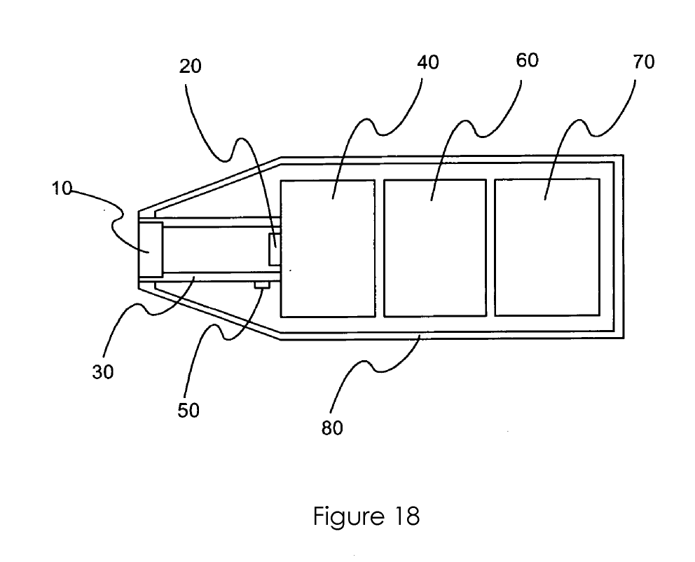

A schematic of a third preferred embodiment of the device is shown in FIG. 18. In this embodiment, the device is contained within a housing 80 that includes an output window 10 through which intense violet-blue light can be delivered to a region of the skin. Prior to the light emission, window 10 is placed in intimate contact with the region of skin to be treated. During the emission, window 10 is held in contact with the skin. After emission, the window can be repositioned to a new region of skin and the treatment can be repeated.

One purpose of window 10 is to transmit the light produced by the light source 20 to the region of the skin to be treated. Therefore, window 10 must be formed of a material transparent to the therapeutic wavelengths produced by light source 20. Sapphire is a preferred material but other transparent materials can be used, including fused quartz, fused silica, polymeric materials, opal glass, or glass. By transparent it is meant that the material has a transmissivity at the therapeutic wavelength of at least 50%, although lower transmissivity can be acceptable for various reasons, including the use of diffusive materials such as opal glass to improve uniformity or eye safety or if the light that is not transmitted on the first pass has additional opportunities for transmission, say, because of a reflector surrounding the light source.

Another purpose of window 10 is to provide a heat sink for the skin so that the skin temperature does not increase to a temperature that is high enough to cause excessive discomfort or damage the skin. Violet-blue light is absorbed within a short distance in skin (effective absorption length of approximately 0 3 mm) and causes the skin temperature to increase. Heat transfer from the skin into window 10 mitigates this temperature rise. A 5 mm thick sapphire disk one centimeter in diameter has enough heat capacity and has a high enough thermal diffusivity to accept 25 Joules/cm² of heat during a 10 second exposure with a temperature increase of less than 20° C. Materials other than sapphire can be used for window 10.

In this embodiment of the invention, window 10 is at or near the nominal skin temperature prior to contact with the skin and does not substantially cool the surface of the skin below its nominal temperature. The nominal skin temperature is the temperature of the skin prior to contact or illumination, and is generally around 32 to 35° C. In this case, the window does not pre-cool the skin but serves as a heat sink during light emission so as to prevent the skin from reaching too high a temperature. In an aspect of the first embodiment, the heatsink would limit the maximum temperature rise in the epidermis to less than about 25° C.

Another aspect of the third embodiment of the invention involves cooling window 10 to a temperature below the nominal skin temperature, for example to a temperature between 0° C. and the nominal skin temperature. When window 10 is placed in contact with the skin prior to light emission, the skin is pre-cooled by the window to lower the skin temperature below the nominal skin temperature. During the light emission, the window 10 provides heat sinking for the skin that is concurrent with the emission.

The most preferred area dimension for this window 10 is about 1 cm² so that small regions of skin like the side of the nose or even individual acne lesions can be treated. In another aspect of the current invention, window 10 can be as large as 5 cm² or even 25 cm² so as to be able to treat a number of lesions or somewhat larger area at a time. However, the maximum size of window 10 is limited by the need for the entire area of the window to be in contact with skin so that it can provide a heat sink to the entire region of skin being illuminated. Too large a window would not conform to the skin where the body is curvaceous, such as regions of skin on or near the nose and upper lip.

The term “spot size” as used in this document refers to the area of the treatment beam at the emitting surface of window 10. The perimeter of this area can be defined by the locations where the intensity of the treatment beam drops to 1/e² of the intensity at the center of the spot. The output window 10 can have a larger size than the spot size in order, for example, to accommodate an optical skin sensor, or can have a different geometry, for example the treatment beam is square and the output window 10 is round for lower cost and ease of manufacturing. In one aspect, the spot size is about 0.81 cm² with a square cross-section and the window is circular with an area of about 1.3 cm².

One aspect of the third embodiment of the invention includes a mixer 30 that is used to make the light emitted by the light source 20 more spatially uniform upon illuminating the skin. It is desirable for the spatial uniformity of the illumination at the skin to have a variation of less than +/−40% so that all of the treated skin receives a similar dose of light. In a preferred aspect, mixer 30 is a hollow aluminum tube with square cross-section about 2 cm in length. The walls of mixer 30 are substantially non-absorbing at the therapeutic wavelengths emitted by source 20 so that light impinging upon the walls of mixer 30 is reflected. As the light travels through mixer 30 from light source 20 to output window 10, the spatial uniformity of the light increases. The length, maximum absorption, and cross-sectional geometry of mixer 30 required for sufficient mixing of the light are dependent upon the size of window 10 and the size and output characteristics of light source 20.

In another aspect, mixer 30 could be a solid light guide in which light from source 20 is totally internally reflected along the light guide to window 10. A mixer that is a solid light guide could itself form the exit aperture for the light and thereby serve as window 10.

In another aspect, it is conceivable that a light source with sufficient uniformity and size could be developed that would make mixer 30 unnecessary.

In an aspect of the third embodiment a two-dimensional array of LED's is used for light source 20. Multiple LED's with optical emission at a wavelength of 405 nm are used to construct a source that delivers about 2.5 Watts of optical power. A 2.5 Watt source delivers about 25 Joules of energy to a 1 cm² region of the skin in 10 seconds. This is approximately equivalent to the dose delivered by the aforementioned ClearLight device in a single 15-minute treatment. Available LED's are currently about 10-15% efficient at converting electrical light to optical power so that about 250 Joules of waste heat is generated for a 25 Joule treatment dose.

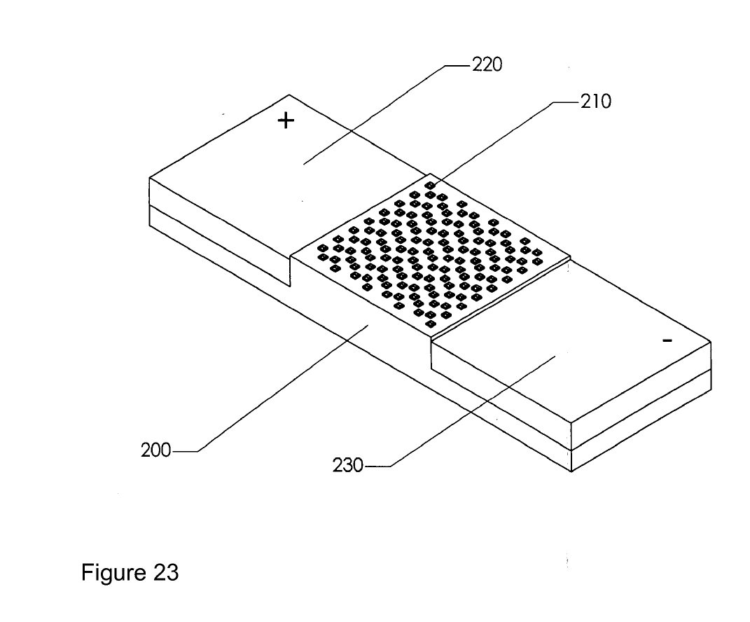

One aspect of a two-dimensional LED light source is shown schematically in FIG. 23. In this aspect, the light source is a two dimensional array of 128 light emitting diode dice 210, such as available from Medical Lighting Solutions, Inc. (Oviedo, Fla.). The dice are the raw semiconductor light-emitting device, by which it is meant that the die are not part of an assembly or package, and therefore do not include lenses. In this application, the foregoing are referred to as “unlensed” LED's. Commercial LED's are often sold as lamp assemblies that include the die, a substrate upon which the die is mounted, electrical leads, and an encapsulation that is shaped to form a lens. In this aspect of the present invention, the dice are bonded to a copper heatsink 200 with thermally conductive epoxy that serves to remove heat from the die when they are energized. Electrical contact to the dice are made with wire-bonds, with 32 parallel strands each having four die connected in series. Each series is wire-bonded to a positively-charged busbar 220 and a negatively-charged busbar 230 such that current flows through the series of four dice. The busbars are electrically isolated from the copper heatsink. This particular configuration uses a supply voltage of approximately 16V. Each die has nominally 4.5 mW of optical output at 405 run with 20 mA of drive current, which provides about 575 mW of intense violet-blue light from the array. The dice can be driven with substantially higher current than 20 mA to yield a light source approaching 2.5 W, without an excessive reduction of lifetime, as long as adequate cooling is provided. Such adequate cooling can take the form of good coupling to the copper heatsink, and even thermally coupling the heat sink to another heat removal element.

In another aspect, violet-blue diode lasers are used as light source 20. For example, Nichia America, Inc. (Mountville, Pa.) manufactures diode lasers with 30 mW of optical output with peak wavelengths available in the 400-415 nm band with 70 mA of drive current (Nichia part no. NDHV310ACA). Therefore, a light source of 100 mW, 500 mW, and 2.5 W of intense violet-blue light can be created by an array of about 3, 16, or 83 laser diodes, respectively. As with the LED's, the laser diodes can be driven with a higher current if well-coupled to an adequate heatsink and/or if a reduction of lifetime is acceptable, reducing the number of diode lasers required. In addition, violet-blue diode lasers are currently in an active area of research with regular performance improvements, making diode lasers an increasingly viable light source in the present invention.

The light source of this embodiment most preferably has an output concentrated in the wavelength band of approximately 400-420 nm which generally matches the absorption peak of the porphyrins believed to be most prevalent in the acne regions. This band also generally matches the in vitro action spectrum reported by Kjeldstad and Johnsson (1986), which has a peak around 412-415 nm. However, the output could also be in a broader wavelength band from 400-450 nm.

The light source preferably has an output power of at least 100 mW/cm² in the violet-blue band, but more preferably has an output power of at least 500 mW/cm² in the violet-blue band.

In still another aspect, alternate constructions of light source 20 can be used. Additional embodiments also emit light energy in wavelength bands in addition to the violet-blue band, such as green or yellow bands that may also have porphyrin absorption or red bands that are believed to have anti-inflammatory benefits.

In the embodiment shown in FIG. 18, mixer 31 also has the function of transferring heat absorbed by output window 11 to a thermal battery 41. The heat transfer of mixer 31 should be high enough to ensure that the heat conducted from the skin and deposited in window 11 during a previous exposure has been substantially removed from window 11 prior to the commencement of a subsequent exposure. In an alternate embodiment of the current invention, the functions of mixer 31, namely light mixing and heat transfer, could be performed by two distinct components. It will also be appreciated by those skilled in the art that such a thermal battery is not required in all embodiments, particularly if a fan or a thermoelectric device is used for cooling.

The illustrated embodiment of the device also employs the use of a temperature sensor 51 to ensure that the assembly comprised of window 11, mixer 31, light source 21, and thermal battery 41 are not at an excessive temperature prior to the commencement of a treatment pulse. An excessive temperature may be reached after several treatment pulses. A temperature sensor is more important in the aspect of the device that cools the window 10 below room temperature prior to illumination. In such an aspect, it may be desirable to have temperature sensor 51 closer to window 11 to ensure the window is at the proper temperature prior to contact with the skin.

The illustrated embodiment of the present invention also has a thermal battery 41 that is composed substantially of a material with sufficient heat capacity as to allow the device to work for tens or hundreds of ten-second pulses with a temperature rise of less than 10° C. This heat removal element can be simply a mass of metal. Alternatively, a material that undergoes a phase change near room temperature can be used. These phase change materials can absorb large amounts of heat with little temperature increase. Optimized materials designed for phase change near room temperature or near skin temperature are available from several manufacturers, such as TEAP Energy (Perth, Australia). These materials can be contained within a metal housing designed to efficiently transfer the heat to the phase change material. Phase change materials with energy densities of about 50 J/cm³° C. are readily available. A thermal battery that accepts the waste heat of over 100 exposures is inexpensive and is easily contained within a hand held device. Another type of thermal battery involves the use of a compressed substance, such as CO₂, which cools upon expansion and can thereby absorb heat energy from a higher temperature source.

A thermal battery 41 of the device can be “re-charged” by simply allowing the device to sit in a room-temperature environment, by placing the device into a refrigerator, or by placing the device in contact with a second device designed to actively conduct heat from thermal battery 41, by replacing or re-pressurizing the compressed substance, or by some other recharging mechanism.

Another aspect of the current invention contains a finned heat sink and fan to more efficiently reject heat from the thermal battery into the room. A heat sink and fan that requires less than 1 Watt and fits into a hand-held device are available from several manufacturers, including Wakefield Thermal Solutions (Pelham, N.H.). Although the finned heatsink can be open to the air outside the housing, the element is to be considered inside the housing.

Still another feature of the current invention is a thermoelectric cooler module, also known as a Peltier-effect device, such as available from Melcor (Trenton, N.J.) to remove heat from thermal battery 41. A device using a thermoelectric cooler module requires a small thermal battery or even no thermal battery at all.

Still another feature of the embodiment is a finned heat sink and fan as a heat removal element to reject heat directly from the device. For example, the light source and the output window can be thermally coupled directly to a finned heatsink that is air-cooled by a fan. Such an aspect operates in a steady-state condition where the device does not need to be thermally recharged and could operate indefinitely from a heat transfer standpoint. This aspect can also use a thermoelectric cooler module.

The embodiment of the invention also contains an electrical battery 61 and control electronics 71. Batteries with energy densities greater than 500 J/cm³ are readily available and a battery that powers the current invention for more than 100 exposures is inexpensive and is easily contained within a hand-held device. An alternative embodiment can be powered from mains power rather than from a battery or battery pack.

It is possible that the light output of some embodiments of the present invention may not be eye safe without mitigation, particularly in the case of diode laser-based light sources. In this event, preferred aspects employ an optical diffuser so that an integrated radiance of the light is reduced to an eye safe value. The diffuser can include a transmissive diffuser, such as PTFE or opal glass, and can include a reflective diffuser, such as Spectralon (Labsphere, Inc., North Sutton, N.H.).

A preferred aspect of the embodiment of the present invention also includes a contact sensor that enables light emission only when the device is in substantial contact with a surface, including the surface of the skin. Most preferably the contact sensor is indicative of contact between the output window 11 and the skin, thereby helping to ensure that the output window 11 provides an effective heatsink for the skin. A contact sensor can also act to reduce emission into the ambient environment that may be uncomfortably bright or may even not be eye safe. A contact sensor can be made of mechanical switches, capacitive switches, piezoelectric materials, or other approaches, and can include sensors located around the periphery of the output window 11. The contact sensor also preferably works only on compliant materials such as skin, so that contact with eyeglasses or flat transparent surfaces would not result in a positive indication of contact. This can be achieved, for example, by recessing the actuation buttons of a contact sensor below the emitting surface of window 21, such that contact with a flat, hard surface would not actuate the buttons. Also most preferably the contact sensor acts as a trigger for light emission, such that light emission is automatically triggered when substantial contact is made with the skin. The light emission can be terminated after a fixed exposure time or if contact is broken or for other reasons. An automatic trigger upon contact is convenient for the user and removes the requirement for a separate trigger, such as one actuated by a finger.

A preferred aspect of a battery-powered embodiment is one in which the battery would directly power the light source in a direct drive configuration. By “directly power” and “direct drive” it is intended to mean that the instantaneous current flowing through the battery and the instantaneous current flowing through the light source at a particular moment in time are substantially equivalent. The instantaneous currents differ only in that a comparatively small amount of current drawn from the battery is used to power the non-light-source components, such as the control electronics.

### Detailed Thermal Calculations

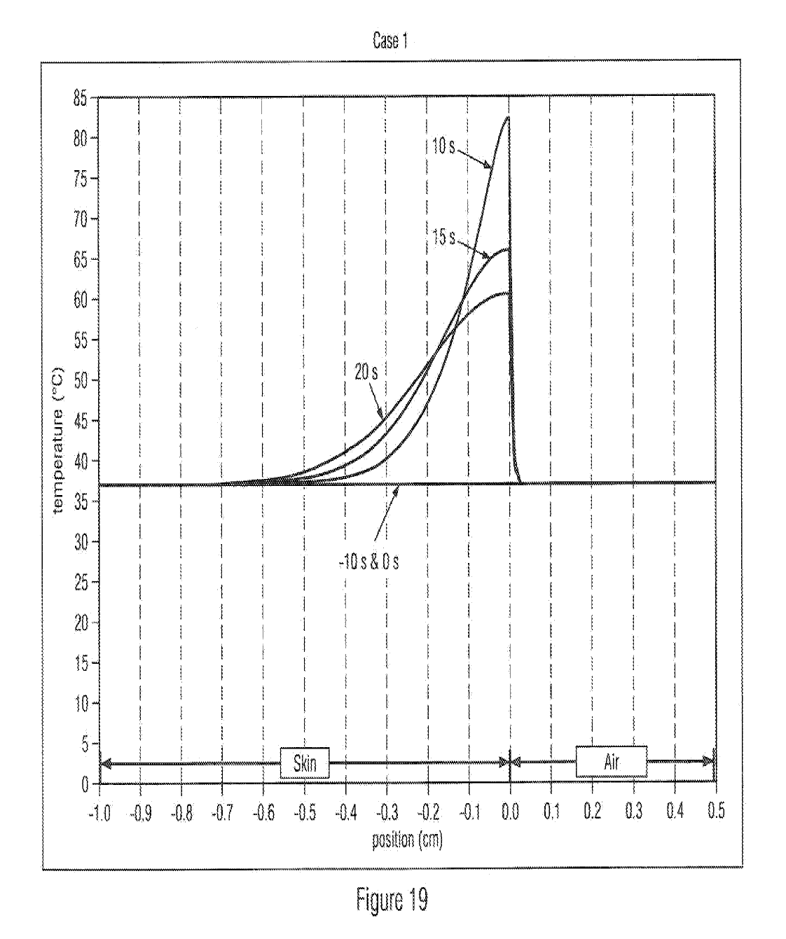

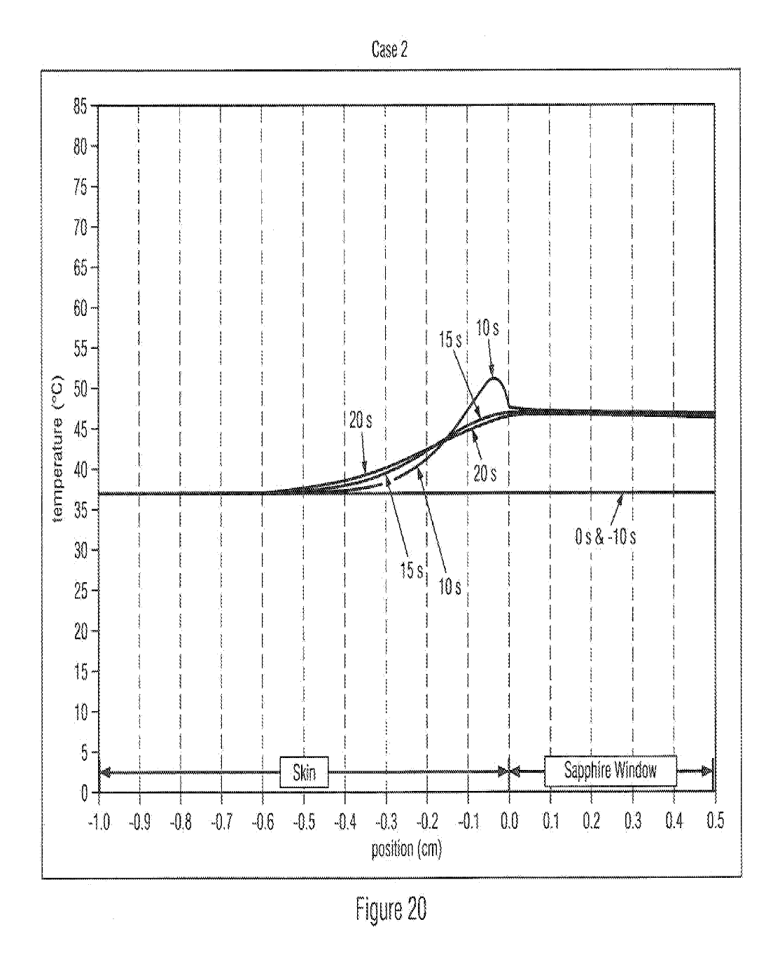

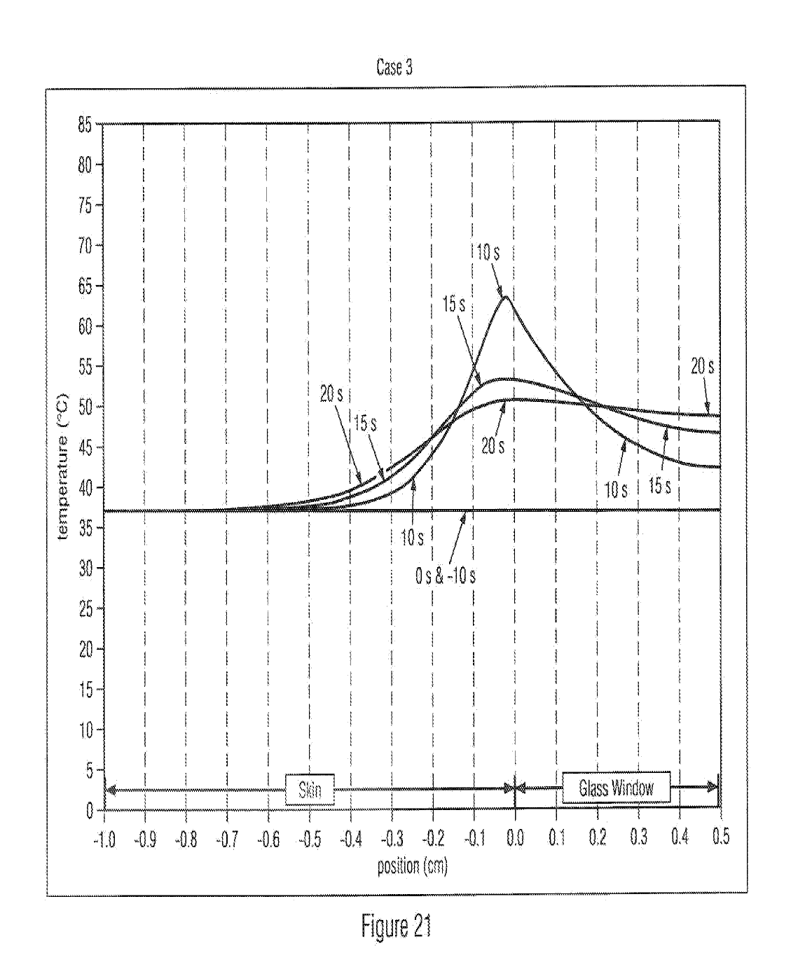

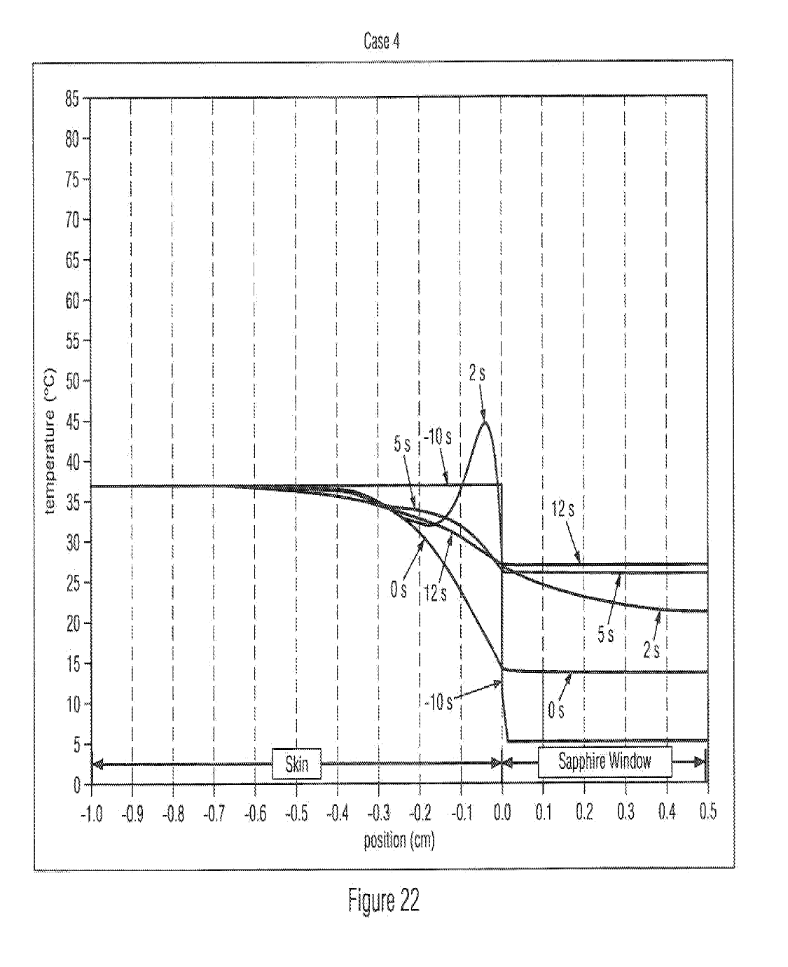

A finite element model of the first embodiment and of skin has been developed to simulate the heat transfer occurring prior to, during, and after light exposure of the skin. Many different cases have been modeled. Four cases have been included with this application. They are labeled Case 1, Case 2, Case 3, and Case 4 and the graphical results are shown in FIG. 19, FIG. 20, FIG. 21, and FIG. 22, respectively. The graphs contained in FIGS. 19-22 show the temperature of the skin and window versus position. Regions to the left of position x=0 are skin. Regions to the right of position x=0 are either air (Case 1) or the window contacting the skin (Case 2, Case 3, and Case 4).

In each case the initial temperature of the skin is 37° C. for the purposes of these calculations. In each case except for the first case, the output window of the device is touched to the skin at time t=−10 s and held in contact with the skin for 10 seconds prior to commencement of illumination of the skin. The first case simulates the treatment where the window is not held in contact with the skin so that there is only air in contact with the skin. In Case 2 and in Case 3, the initial temperature of the window is 37° C., representing the nominal skin temperature. In Case 4, the initial temperature of the window is 5° C. In each case, commencement of illumination occurs at time t=0 s. For cases 1, 2, and 3, the skin is illuminated with light for 10 s at an intensity of 2.5 W/cm². In the fourth case, the skin is illuminated for 2 s at an intensity of 12.5 W/cm². In each case an effective absorption length in skin of 0.3 mm was used to model the absorption of the incident light. This effective absorption length, 0.3 mm, is approximately that of 405 nm light in skin.

Notice from the graph of the results for Case 1 shown in FIG. 19 that when only air is in contact with the skin, the temperature of the skin reaches a maximum temperature of over 80° C. A temperature of 80° C. is above the threshold for damage to the skin and is painful.

The graph of the results for Case 2 in FIG. 20 shows that when a sapphire window with thickness of 5 mm and initial temperature of 37° C. placed in contact with the skin for 10 prior to the pulse of illumination, the maximum temperature of the skin is only approximately 52° C. This temperature is below the threshold for damage to the skin. It is perceived as hot but easily tolerated with little or no pain.

The graph of the results for Case 3 in FIG. 21 shows that a glass window with thickness of 5 mm and initial temperature of 37° C. does not perform as well as sapphire because of the limited thermal diffusivity of the glass. Notice the large temperature gradient in the glass window that existed at time, t=10 s, indicating that heat was not effectively transferred to the back surface of the glass during the illumination pulse. The maximum temperature of the skin in Case 3 is approximately 63° C.

Finally, the graph of the results for Case 4 in FIG. 22 shows that by cooling a sapphire window to 5° C. prior to contacting the skin, the maximum temperature of the skin is less than 45° C. even though the illumination of 12.5 W/cm² is much more intense than in the previous three cases.

From these simulations it is evident that a device with an output window placed in contact with the skin prior to or during the exposure of skin is effective at preventing thermal injury to the skin.

It will be appreciated that the method, system and apparatus taught herein can effectively reduce the level of colonization of a patient's skin by the P. acnes bacteria. By treating the affected areas as discussed above, the concentration of bacteria in the sebaceous ducts and glands can be significantly reduced. Lower bacterial load reduces the concentration of inflammatory bacterial metabolites, thereby reducing the likelihood of the induction of an inflammatory cascade of the type that produces lesions. Essentially, by breaking the chain of the inflammatory cycle, the present invention reduces and prevents the formation of lesions, and/or can enhance the rate at which lesions clear.

Stated differently, some embodiments of the present disclosure use selective photothermolysis of the pilosebaceous duct, gland and/or contents. It has been determined that the bulk of the material within an infected gland is composed of P. acnes bacteria. This allows selective targeting of absorbing chromophores produced by the bacteria, rather than the sebum produced by the sebaceous gland. This also provides the possibility of delivering a sufficient dose to the affected area within an acceptably short time. The result is a treatment regimen that may also involve reduction of hyperkeritinization, bacterial destruction, and reduction of inflammation. In addition, the ability of the sebaceous gland to prevent leakage of its content into the surrounding dermis may be increased through dietary supplementation of GLA or similar long-chain fatty acids which are typically deficient in acne sufferers.

### Methods of Therapy

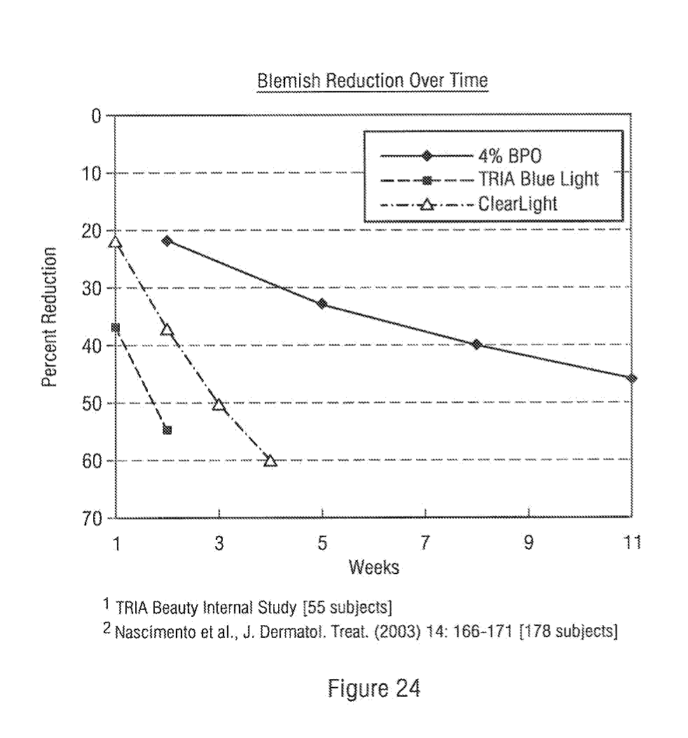

FIG. 24 illustrates an example embodiment of a phototherapy regime in accordance with the present disclosure. For example, a target portion of a subject's skin may be contacted with a cleanser composition (e.g., for about one minute to about ten minutes), illuminated with blue light (e.g., about 390 nm to about 430 nm), and contacted with an anti-oxidant serum (e.g., for about one minute to about ten minutes). In some embodiments, a target portion of a subject's skin may be rinsed after contact with a cleanser composition and/or after contact with an anti-oxidant composition. Each cycle of cleanser, light, anti-oxidant treatments may be repeated in whole or in part. For example, a subject may perform once per day every day and/or every other day. Cycles may be repeated (e.g., once per day) for up to about ten days, up to about 20 days, up to about 30 days, up to about 40 days, up to about 50 days, up to about 60 days, and/or up to about 90 days. In some embodiments, a regime may be practiced for in two phases. For example, during a first phase, compositions may be administered (i) at higher concentrations, (ii) for greater times per application, and/or (iii) more frequently until a desired reduction in symptoms is observe (e.g., a substantial reduction) and, then scaled back (e.g., in one step or a series of steps) to a maintenance level during a second phase.

The compositions, devices, systems, and/or methods of the disclosure may be useful for skin care. Conditions that may be improved by compositions, devices, systems, and/or methods of the disclosure may include, for example, acne vulgaris (e.g., mild, moderate, and severe), acne conglobata, acne rosacea, melasma, acne of pregnancy and other similar dermatological conditions.

The dosing of cleansing and anti-oxidant compositions will depend on absorption, inactivation, and excretion rates of the respective compositions as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition.

In some embodiments, compositions, devices, systems, and/or methods of the disclosure may be used with a given subject at a point in time or over a period of time. For example, a regime including cleanser, blue light, and anti-oxidant serum may be repeated once per day over a period of 2 days to 3 months (e.g., one week, two weeks, three weeks, four weeks, five weeks, six weeks, seven weeks, eight weeks, nine weeks, ten weeks, eleven weeks, and/or twelve weeks).

Successful use of a skin care regime in a subject may be determined by one of ordinary skill in the art using one or more possible metrics, including but not limited to, a reduction in lesion size, lesion number (e.g., decrease in formation and/or increase in resolution), and/or lesion type. For example, a metric of success may include the rate at which papules and pustules (inflammatory acne) clear. In some embodiments, a regime (e.g., an 8-week regime) may achieve a clearance rate of up to about 25%, up to about 30%, up to about 35%, up to about 40%, or up to about 45%. Appearance and/or clearance of whiteheads and blackheads (non-inflammatory acne) may be measured.

Improvement relative to no treatment or to another treatment may be scaled as needed or desired. For example, improvement may be assessed on a 5-point scale in which 0 is no improvement, 1 is minimal improvement, 2 is mild improvement, 3 is moderate improvement, 4 is good improvement, and 5 is excellent improvement. In some embodiments, a regime (e.g., an 8-week regime) may produce an improvement over untreated skin of from about 0.5 to about 5.

As will be understood by those skilled in the art who have the benefit of the instant disclosure, other equivalent or alternative compositions, devices, methods, and systems for skin care can be envisioned without departing from the description contained herein. Accordingly, the manner of carrying out the disclosure as shown and described is to be construed as illustrative only.

Persons skilled in the art may make various changes in the shape, size, number, and/or arrangement of parts without departing from the scope of the instant disclosure. For example, the position and number of light sources, filters, optical mixers, switches, power supplies, and output windows may be varied. In some embodiments, one or more parts may be interchangeable. Interchangeability may allow the intensity and/or wavelength of light emitted to be custom adjusted (e.g., by tuning the light source, optical filters, or both). In addition, the size of a device and/or system may be scaled up (e.g., to be used for adult subjects) or down (e.g., to be used for juvenile subjects) to suit the needs and/or desires of a practitioner. Where the verb “may” appears, it is intended to convey an optional and/or permissive condition, but its use is not intended to suggest any lack of operability unless otherwise indicated. Also, where ranges have been provided, the disclosed endpoints may be treated as exact and/or approximations as desired or as demanded by the particular embodiment. Where the endpoints are approximate, the degree of flexibility may vary in proportion to the order of magnitude of the range. For example, a range of endpoint of “about 50” may one the one hand include 50.5, but not 52.5 or 55 in the context of a range of about 5 to about 50 and, on the other hand, include 55, but not 60 or 75 in the context of a range of about 0.5 to about 50. In addition, it may be desirable, in some embodiments, to mix and match range endpoints. Also, in some embodiments, each figure disclosed (e.g., in one or more of the Examples and/or Drawings) may form the basis of a range (e.g., +/− about 10%, +/− about 100%) and/or a range endpoint. According to some embodiments, expressions of a concentration of a material of “up to” (e.g., up to about 10%) includes, at the lower end of the range, any amount of the material greater than zero. Persons skilled in the art may make various changes in methods of preparing and using a composition, device, and/or system of the disclosure. For example, a composition, device, and/or system may be prepared and or used as appropriate for animal and/or human use (e.g., with regard to sanitary, infectivity, safety, toxicity, biometric, and other considerations).

All or a portion of a device and/or system for skin care may be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1

### Anti-Acne Foaming Cleanser

Table 1 below provides an example embodiment of a formulation of an anti-acne foaming cleanser.

TABLE 1

| INCI Name | W/W % |
|---|---|
| Water | 53.710260 |
| Butylene Glycol | 15.000000 |
| Cocamidopropyl Betaine | 7.000000 |
| Sodium C14-16 Olefin Sulfonate | 5.000000 |
| Polysorbate 20 | 5.000000 |
| Glycolic Acid | 4.996800 |
| Sodium Coco-Sulfate | 4.000000 |
| Salicylic Acid | 2.000000 |
| Ethoxydiglycol | 1.500000 |
| Water Lily CE-50045 | .800000 |
| Ammonium Hydroxide | .600000 |
| Linoleamidropropyl PG-Dimonium Chloride Phosphate | .137500 |
| Disodium EDTA | .100000 |
| Menthyl Lactate | .100000 |
| Menthol | .050000 |
| Methylchloroisothiazolinone | .001080 |
| Methylisothiazolinone | .000360 |
| Total: | 100.000 |

### Example 2

### Anti-Acne Antioxidant Serum

Table 2 below provides an example embodiment of a formulation of a anti-acne antioxidant serum.

TABLE 2

| INCI Name | W/W % |
|---|---|
| Water | 65.160000 |
| Alcohol Denat. | 12.500000 |
| Butylene Glycol | 4.500000 |
| Dimethincone/Divinyldimethicone/Silsesquioxane | 3.000000 |
| Crosspolymer | |
| Saccharomyces/Xylinum/Black Tea Ferment | 2.655000 |
| Cyclopentasiloxane | 2.550000 |
| Hydroxyethyl Acrylate/Sodium Acryloyldimethyl | 1.295000 |
| Taurate Copolymer | |
| Salicylic Acid | 1.250000 |
| PEG-40 Stearate | 1.000000 |
| Daucus Carota Sativa (Carrot) Root | .900000 |
| Squalane | .875000 |
| Phytic Acid | .750000 |
| Niacinamide | .500000 |
| Water Lily CE-50045 | .500000 |
| Dimethiconol | .450000 |
| Xanthan Gum | .430000 |
| Glycerin | .300000 |
| Superoxide Dismutase | .200000 |
| Polysorbate 60 | .175000 |
| Potassium Sorbate | .150000 |
| Ammonium Hydroxide | .150000 |
| Sodium Benzoate | .150000 |
| Tetrahexyldecyl Ascorbate | .100000 |
| Disodium EDTA | .100000 |
| Retinyl Palmitate | .100000 |
| Azelaic Acid | .080000 |
| Lecithin | .050000 |
| Hydroxyethylcellulose | .045000 |
| Atelocollagen | .030000 |
| Sodium Chondroitin Sulfate | .030000 |
| Boerhavia Diffusa Root Extract | .025000 |
| Total: | 100.000000 |

### Example 3

### Blue Light Therapy

The efficacy of an example embodiment of a combination topical/blue light phototherapy was assessed in a clinical study. Enrolled subjects (n=30) were diagnosed with mild to moderate acne. A preliminary evaluation was performed noting the nature, number, and size of lesions present. Subjects followed the daily treatment protocol shown in Table 3 below for eight weeks.

TABLE 3
Daily Treatment Protocol

| Time | Treatment | Area A | Area B |
|---|---|---|---|
| Morning | Wash face with non- | | |
| | medicated cleanser | | |
| | Treat with blue light | A 3 cm × 5 cm area is | The rest of the face, |
| | device | treated for 3 minutes | excluding Treatment |
| | | (sweeping/painting | Area A, is treated for |
| | | motion within the | 3 minutes |
| | | 3 cm × 5 cm area). | (sweeping/painting |
| | | Option. During the | motion). |
| | | first two (2) weeks | Option. During the |
| | | only, subjects may | first two (2) weeks |
| | | optionally dwell on | only, subjects may |
| | | each bothersome | optionally dwell on |
| | | lesion for 30 seconds | each bothersome |
| | | within the same 3 cm × | lesion for 30 seconds |
| | | 5 cm area. | within the same 3 cm × |
| | | | 5 cm area. |
| Evening | Wash face with non- | | |
| | medicated cleanser | | |
| | Treat with blue light | A 3 cm × 5 cm area is | The rest of the face, |
| | device | treated for 3 minutes | excluding Treatment |
| | | (sweeping/painting | Area A, is treated for |
| | | motion within the | 3 minutes |
| | | 3 cm × 5 cm area) | (sweeping/painting |
| | | Option. During the | motion). |
| | | first two (2) weeks | Option. During the |
| | | only, subjects may | first two (2) weeks |
| | | optionally dwell on | only, subjects may |
| | | each bothersome | optionally dwell on |
| | | lesion for 30 seconds | each bothersome |
| | | within the same 3 cm × | lesion for 30 seconds |
| | | 5 cm area. | within the same 3 cm × |
| | | | 5 cm area. |

Subjects were re-evaluated weekly during treatment for efficacy (reduction in inflammatory lesions, reduction in non-inflammatory lesions), safety, and subject satisfaction (weeks, 1, 2, 3, 4, 6, and 8). No serious adverse events we reported during the study. Blue light was well tolerated.

During the first two weeks, the estimated daily dose is 53 J/cm² in Area A and 26 J/cm² in Area B. During weeks 3-8 (without dwelling on lesions), the estimated daily dose is 29 J/cm² in Area A and 2 J/cm² in Area B.

Reductions in inflammatory lesions were observed as early as one week into the study and continued through week 8. For example, inflammatory lesions were reduced from baseline an average of 41% in Area A and 32% in area B at week 4. Inflammatory lesions were reduced from baseline an average of 54% in Area A and 44% in area B at week 8.

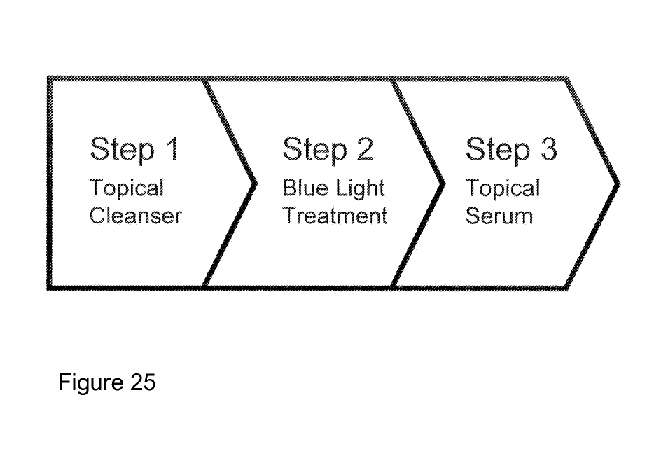

In another clinical trial, subjects (n=55) were exposed to (a) blue light], (b) white light, or (c) a composition comprising 4% (w/w) benzyol peroxide (BPO). Subjects were re-evaluated weekly during each treatment regime and results are illustrated in FIG. 25. The number of skin blemishes observed on subjects who received blue light therapy dropped by over 35% in the first week and by 55% in the second week. By comparison, the number of skin blemishes observe on subjects who received BPO dropped by just over 20% by the second week and by only 45% after eleven weeks.

### Example 4

### Combination Blue Light Therapy

The efficacy of an example embodiment of a combination topical/blue light phototherapy was assessed in a clinical study. Enrolled subjects (n=30) were diagnosed with mild to moderate acne. A preliminary evaluation was performed noting the nature, number, and size of lesions present. Subjects followed the daily treatment protocol shown in Table 4 below for eight weeks.

TABLE 4
Daily Treatment Protocol

| Time | Treatment | Area A | Area B |
|---|---|---|---|
| Morning | Wash face with one | | |
| | (1) pump of foam | | |
| | cleanser* | | |
| | Treat with blue light | A 3 cm × 5 cm area is | The rest of the face, |
| | device | treated for 3 minutes | excluding Treatment |
| | | (sweeping/painting | Area A, is treated for |
| | | motion within the | 3 minutes |
| | | 3 cm × 5 cm area). | (sweeping/painting |
| | | Option. During the | motion). |
| | | first two (2) weeks | Option. During the |
| | | only, subjects may | first two (2) weeks |
| | | optionally dwell on | only, subjects may |
| | | each bothersome | optionally dwell on |
| | | lesion for 30 seconds | each bothersome |
| | | within the same 3 cm × | lesion for 30 seconds |
| | | 5 cm area. | within the same 3 cm × |
| | | | 5 cm area. |
| Evening | Wash face with one | | |
| | (1) pump of foam | | |
| | cleanser* | | |
| | Treat with blue light | A 3 cm × 5 cm area is | The rest of the face, |
| | device | treated for 3 minutes | excluding Treatment |
| | | (sweeping/painting | Area A, is treated for |
| | | motion within the | 3 minutes |
| | | 3 cm × 5 cm area) | (sweeping/painting |
| | | Option. During the | motion). |
| | | first two (2) weeks | Option. During the |
| | | only, subjects may | first two (2) weeks |
| | | optionally dwell on | only, subjects may |
| | | each bothersome | optionally dwell on |
| | | lesion for 30 seconds | each bothersome |
| | | within the same 3 cm × | lesion for 30 seconds |
| | | 5 cm area. | within the same 3 cm × |
| | | | 5 cm area. |
| | Apply one (1) pump | | |
| | of serum† | | |

*Cleanser: 2% salicylic acid and 5% glycolic acid (Example 1).
†Serum: 1.2% salicylic acid (Example 2.

Subjects were re-evaluated weekly during treatment for efficacy (reduction in inflammatory lesions, reduction in non-inflammatory lesions), safety, and subject satisfaction (weeks, 1, 2, 3, 4, 6, and 8). No serious adverse events we reported during the study. Both the blue light and topicals (cleanser and serum) were well tolerated.

During the first two weeks, the estimated daily dose is 53 J/cm² in Area A and 26 J/cm² in Area B. During weeks 3-8 (without dwelling on lesions), the estimated daily dose is 29 J/cm² in Area A and 2 J/cm² in Area B.

Reductions in inflammatory lesions were observed as early as one week into the study and continued through week 8. For example, inflammatory lesions were reduced from baseline an average of 55% in Area A and 49% in area B at week 4. Reductions in non-inflammatory lesions were also observed during the early weeks of treatment as well. For example, non-inflammatory lesions were reduced from baseline an average of 53% at week 6, similar to the clearance rates of inflammatory lesions at week 4.

Inflammatory lesion reductions with the device alone provide clinical significant results at week 4 as well; however, the use of therapeutic topicals provide and approximately a 35% incremental improvement at week 4.

1-41. (canceled)

42. A skin care method comprising:
applying a cleansing composition to a target area of skin, the cleansing composition comprising a first set of antioxidant components;
removing the cleansing composition from the skin;
after removing the cleansing composition from the skin, delivering radiation in a wavelength range between about 390 nm to about 430 nm to the target area of skin, the radiation interacting with the skin to generate intracellular reactive oxygen species (ROS); and
after delivering the radiation, applying an anti-oxidant composition to the target area of skin, the anti-oxidant composition comprising a second set of antioxidant components partially different than the first set of antioxidant components;
wherein the cleansing composition and the anti-oxidant composition include at least one same antioxidant component; and
wherein the anti-oxidant composition includes at least one antioxidant component not included in the cleansing composition.

43. The skin care method of claim 42, wherein the cleansing composition and the anti-oxidant composition include at least one same antioxidant component in different concentrations.

44. The skin care method of claim 42, wherein both the cleansing composition and the anti-oxidant composition include salicylic acid, but in different concentrations.

45. The skin care method of claim 42, wherein the cleansing composition contains a higher concentration of at least one antioxidant component than the anti-oxidant composition.

46. The skin care method of claim 42, wherein the first set of antioxidant components of the cleansing composition comprises at least 2% of the cleansing composition, and the second set of antioxidant components of the cleansing composition comprises at least 2% of the anti-oxidant composition.

47. The skin care method of claim 42, wherein the anti-oxidant composition includes three different antioxidant components.

48. The skin care method of claim 42, wherein the anti-oxidant composition includes five different antioxidant components.

49. The skin care method of claim 42, wherein the first set of antioxidant components comprise at least 5% of the anti-oxidant composition.

50. The skin care method of claim 42, wherein the anti-oxidant composition includes superoxide dismutase (SOD).

51. The skin care method of claim 42, wherein the anti-oxidant composition includes squalene, vitamin B niacinamide, salicylic acid, and superoxide dismutase (SOD).

52. The skin care method of claim 42, wherein the anti-oxidant composition includes black tea ferment.

53. The skin care method of claim 42, wherein the cleansing composition includes an effective exfoliating amounts of at least one of an alpha hydroxy acid and a beta hydroxy acid.

54. The skin care method of claim 42, wherein the radiation is delivered with a light intensity of between 300 and 700 mW/cm² at the target area of skin.

55. A skin care method comprising:
delivering radiation in a wavelength range between about 390 nm to about 430 nm to a target area of skin at an intensity of between 300 and 700 mW/cm², the radiation interacting with the skin to generate intracellular reactive oxygen species (ROS); and
after delivering the radiation to the skin, applying an anti-oxidant composition to the target area of skin, the anti-oxidant composition comprising three different antioxidant components

56. The skin care method of claim 55, wherein the anti-oxidant composition includes five different antioxidant components.

57. The skin care method of claim 55, wherein the first set of antioxidant components comprise at least 5% of the anti-oxidant composition.

58. The skin care method of claim 55, wherein the anti-oxidant composition includes squalene, vitamin B niacinamide, salicylic acid, and superoxide dismutase (SOD).

59. The skin care method of claim 55, wherein the anti-oxidant composition includes black tea ferment.
